(19) United States
(12) Patent Application Publication
(10) Pub. No.: US 2021/0100973 A1
(43) Pub. Date: Apr. 8, 2021

(54) MOBILE METHOD FOR IMPROVING WELLNESS

(71) Applicant: UNGRIPPED LLC, Madison, NJ (US)

(72) Inventor: BRETT LEWIS, MADISON, NJ (US)

(73) Assignee: UNGRIPPED LLC, Madison, NJ (US)

(21) Appl. No.: 16/591,315

(22) Filed: Oct. 2, 2019

(51) Int. Cl. A61M 21/02

(52) U.S. Cl. CPC A61M 21/02; A61M 2021/0044

(57) ABSTRACT

The described invention relates to a mobile application for mobile devices or computers alone or with attachments that deliver sensory stimulation effective to improve a subject's perception of wellness in real time.

### FIELD OF THE INVENTION

The described invention relates generally to mobile approaches for improving emotional responses, mental responses, physiological responses, and more particularly to bilateral sensory stimulation.

### BACKGROUND OF THE INVENTION

### Stress

Stress is the body's normal response to physical, mental, or emotional changes, situations, and forces (Larson K, Healthcommunities.com, 2007). Stress can result from external factors (e.g., events, environment) or from internal factors (e.g., expectations, attitudes, feelings). Stress often occurs in response to situations that are perceived as being difficult to handle or threatening. Common causes for stress (termed stressors) include illness, injury, fear, and anxiety. Each person reacts to stress differently. A healthy response to stress begins quickly; is appropriate in degree and in length; and can improve function, motivation, and productivity (Id.).

The body's response to stress is initiated and controlled by the central nervous system (CNS, made up of the brain and spinal cord). In response to a stressful situation, the brain first signals the inner portion of the adrenal gland (called the adrenal medulla) to release hormones (e.g., adrenaline, noradrenaline) that initiate the “flight-or-fight” response. Next, the brain triggers the release of other hormones, such as cortisol, to sustain this response. Effects of these hormones include changes in digestion (caused by increased blood flow to the muscles and reduced blood flow to the gastrointestinal tract); dilation of blood vessels; increased breathing (respiration) rate; increased heart rate and blood pressure (caused by increased blood flow to the brain); and increased blood sugar levels (to provide more energy) (Id.).

### Types of Stress

Stress can be acute (short term), episodic (frequent), or chronic (long term). Acute stress, which is the most common type, results from events, pressures, and demands of the recent past and anticipated in the near future. Acute stress that occurs frequently is called episodic stress. This type of stress is common in people whose lives are extremely busy or disorganized, and in those who tend to worry excessively (Id.). A variety of negative feelings can occur from real or perceived stressors, such as fear, doubt, unease, frustration, anger, and guilt. According to the Gallup 2019 Global Emotions Report, about a third of people worldwide were stressed, worried, and in pain last year, and more than half of Americans regularly feel pressure and strain (available at https://www.gallup.com/analytics/248906/gallup-global-emotions-report-2019.aspx).

Chronic stress, which occurs when a stressful situation is prolonged and continuous, is a serious condition. It often causes severe physical and emotional symptoms, including anxiety and depressive disorders (Larson K, Healthcommunities.com, 2007). One extreme form of stress, posttraumatic stress disorder (PTSD), is an anxiety disorder that can occur following life-threatening events, such as military action, natural disasters, terrorist incidents, serious accidents, and violent personal assaults (e.g., rape). Following severe trauma, some stress reactions do not go away and even worsen over time. People who suffer from PTSD often relive the experience through nightmares and flashbacks, have difficulty sleeping, and feel detached or estranged. These symptoms are often severe and long lasting and they can significantly impair daily life.

### Physiological Responses to Stress

Chronic stress can have many maladaptive (unhealthy) effects on the body.

Brain. Chronic stress is linked to macroscopic changes in volume variations and physical modifications of neuronal networks in certain brain areas such as the prefrontal cortex (PFC) and the limbic system (including the amygdala, thalamus, and hippocampus) (Mariotti A. Future Sci OA. 2015 November; 1(3): F5023). The amygdala is a key region of the brain involved in emotional learning, particularly in the acquisition and expression of fear and anxiety in response to traumatic or stressful events (Girgenti M J, et al. Curr Psychiatry Rep. 2017 Sep. 25; 19(11): 85).

Immune system. Psychological stress can induce an acute response in the immune system commonly associated with infections and tissue damage, and increase the levels of circulating cytokines and various biomarkers of inflammation (Mariotti A. Future Sci OA. 2015 November; 1(3): FSO23, citing Black P H. Brain Behay. Immun. 2002; 16(6):622-653). In chronic stress, the immune system can be overstimulated, causing imbalances that establish a state of chronic low-grade inflammation, a possible prelude to various illnesses. Diseases whose development has been linked to both stress and inflammation include cardiovascular dysfunctions, diabetes, cancer, and autoimmune syndromes (Id.).

Musculoskeletal system. With sudden onset stress, the muscles tense up all at once, and then release their tension when the stress passes. Chronic stress causes the muscles in the body to be in a more or less constant state of guardedness. When muscles are taut and tense for long periods of time, this may trigger other reactions of the body and even promote stress-related disorders. For example, both tension-type headache and migraine headache are associated with chronic muscle tension in the area of the shoulders, neck and head. Musculoskeletal pain in the low back and upper extremities has also been linked to stress, especially job stress. Millions of individuals suffer from chronic painful conditions secondary to musculoskeletal disorders (American Psychological Association, https://www.apa.org/helpcenter/stress-body).

Cardiovascular system. The heart and blood vessels comprise the two elements of the cardiovascular system that work together in providing nourishment and oxygen to the organs of the body. The activity of these two elements is also coordinated in the body's response to stress. Acute stress (meaning stress that is momentary or short-term such as meeting deadlines, being stuck in traffic or suddenly slamming on the brakes to avoid an accident) causes an increase in heart rate and stronger contractions of the heart muscle, with the stress hormones adrenaline, noradrenaline and cortisol acting as messengers for these effects. In addition, the blood vessels that direct blood to the large muscles and the heart dilate, thereby increasing the amount of blood pumped to these parts of the body and elevating blood pressure. Once the acute stress episode has passed, the body usually returns to its normal state.

Chronic stress can contribute to long-term problems for heart and blood vessels. The consistent and ongoing increase in heart rate, and the elevated levels of stress hormones and of blood pressure, can take a toll on the body, and increase the risk for hypertension, heart attack or stroke. Repeated acute stress and persistent chronic stress may also contribute to inflammation in the circulatory system, particularly in the coronary arteries; this is one pathway that is thought to tie stress to heart attack. It also appears that how a person responds to stress can affect cholesterol levels.

Respiratory system. Stress and strong emotions can present with respiratory symptoms, such as shortness of breath and rapid breathing, as the airway between the nose and the lungs constricts. For people without respiratory disease, this is generally not a problem as the body can manage the additional work to breathe comfortably, but psychological stressors can exacerbate breathing problems for people with pre-existing respiratory diseases, such as asthma and chronic obstructive pulmonary disease (COPD; includes emphysema and chronic bronchitis). Some studies show that an acute stress—such as the death of a loved one—can actually trigger asthma attacks. In addition, the rapid breathing—or hyperventilation—caused by stress can bring on a panic attack in someone prone to panic attacks (Id.).

### Techniques for Stress Reduction

People cope with stressful situations in different ways. Some people rage, some people simply shrug them off, some people avoid situations that provoke those feelings, some people deny those feelings.

For example, for people that commute to work by public transportation, some people read, some people listen to music, some people stare out the windows of a car or bus, some sleep, some do work, some try to avoid the crowds.

There are many other ways people cope with stress, both healthy and unhealthy. Unhealthy methods include substance abuse, addictive behavior, social withdrawal, procrastination, repression, overextension, brooding, aggression, self-harm, and hypersomnia. Healthier methods may include behavioral therapy, pharmaceutical therapy, and avoiding stressful situations. One type of behavioral therapy for PTSD, called eye movement desensitization and reprocessing (EMDR), encourages a patient to focus on a trauma memory while simultaneously undergoing eye movements in order to reduce the emotion associated with the trauma memories. However, EMDR is still a controversial treatment. Although the eye movements are integral to the basic procedure, there is little evidence that the movements have any impact on outcome measures (Davidson P R, Parker K C H. J Consult Clin Psychol. 2001 April; 69(2): 305-16, citing Cahill S P, et al. J Anxiety Disord. 1999 January-April; 13(1-2): 5-33).

Another type of coping mechanism is activation of the Autonomous Sensory Meridian Response, or “ASMR.” ASMR is a perceptual sensory phenomenon that induces pleasant physiological responses, such as transient paresthesia, and chronic pain relief, in people who are able to experience it, as well as other mental and emotional responses, such as relaxation, stress reduction, insomnia relief, and mood boosting responses. ASMR is trigged by certain auditory, visual and somatic stimulation, such as light tickling, whispering, crisp sounds, such as chewing or fingernail tapping, and the like. Like EMDR, the use of ASMR is controversial.

The described invention provides methods and systems for increasing a person's ability to manage stress by delivering sensory stimulation and the refocusing the person's attention on visual, auditory, somatic, olfactory, and/or vestibular sensations that are effective to improve a person's perception of wellness by reducing negative emotional, mental and physiological responses and/or increasing positive emotional, mental, and physiological responses.

### BRIEF SUMMARY OF THE INVENTION

The described invention provides a method for improving a perception of wellness by a subject comprising: providing a wellness improving mobile application, wherein the mobile application is configured to communicate with a mobile device or attachment thereof and is designed to deliver at least one sensory stimulation, delivering the at least one sensory stimulation designed to deliver the sensory stimulation to the subject, and the subject interacting with the mobile application, and the interaction in (c) improving the perception of wellness by the subject; wherein the at least one sensory stimulation comprises visual stimulation, auditory stimulation, somatic stimulation, olfactory stimulation, and vestibular stimulation.

According to some embodiments, the mobile application is designed to deliver the at least one sensory stimulation via continuous delivery, intermittent delivery, static delivery, kinetic delivery, unilateral delivery, bilateral delivery, unilongitudinal delivery, bilongitudinal delivery, unicoronal delivery, and bicoronal delivery or any combination thereof.

According to some embodiments, the delivery of the at least one sensory stimulation is in a pattern, a cycle, a sequence or any combination thereof.

According to some embodiments of the method, the interaction in (c) comprises actively, passively, or physiologically tailoring the type or amount of sensory stimulation delivered or both, the type or amount of method of delivery of the sensory stimulation delivered or both, the order or the amount of patterns delivered or both, the order or the amount cycles delivered or both, and the order or amount of sequences delivered or both, or any combination thereof.

According to some embodiments, the improving of the perception of wellness by the subject comprises reducing and/or alleviating a level of one or more negative feelings felt by a subject, invoking, increasing, and/or maintaining a level of one or more positive feelings felt by a subject; reducing and/or alleviating a negative mood felt by a subject, invoking, increasing, and/or maintaining a positive mood felt by a subject, inducing the prevalence or occurrence of one or more desirable physiological responses, reducing the prevalence or occurrence of one or more undesirable physiological responses, or any combination thereof.

According to some embodiments, the at least one sensory stimulation comprises visual stimulation; or the at least one sensory stimulation comprises auditory stimulation; or the at least one sensory stimulation comprise somatic stimulation; or the at least one sensory stimulation comprises olfactory stimulation; or the at least one sensory stimulation comprises vestibular stimulation; or the at least one sensory stimulation comprises visual and auditory stimulation; or the at least one sensory stimulation comprises visual and somatic stimulation; or the at least one sensory stimulation comprises visual and olfactory stimulation; or the at least one sensory stimulation comprises visual and vestibular stimulation; or the at least one sensory stimulation comprises auditory and somatic stimulation; or the at least one sensory stimulation comprises auditory and olfactory stimulation; or the at least one sensory stimulation comprises auditory and vestibular stimulation; or the at least one sensory stimulation comprises somatic and olfactory stimulation; or the at least one sensory stimulation comprises somatic and vestibular stimulation; or the at least one sensory stimulation comprises olfactory and vestibular stimulation.

According to some embodiments, the mobile application is designed to deliver the at least one sensory stimulation via bilateral delivery.

According to some embodiments, the mobile application is designed to deliver the at least one sensory stimulation via intermittent bilateral delivery.

### BRIEF DESCRIPTION OF THE DRAWINGS

FIG. 1 shows a flow diagram that describes the delivery of a sensory stimulation.

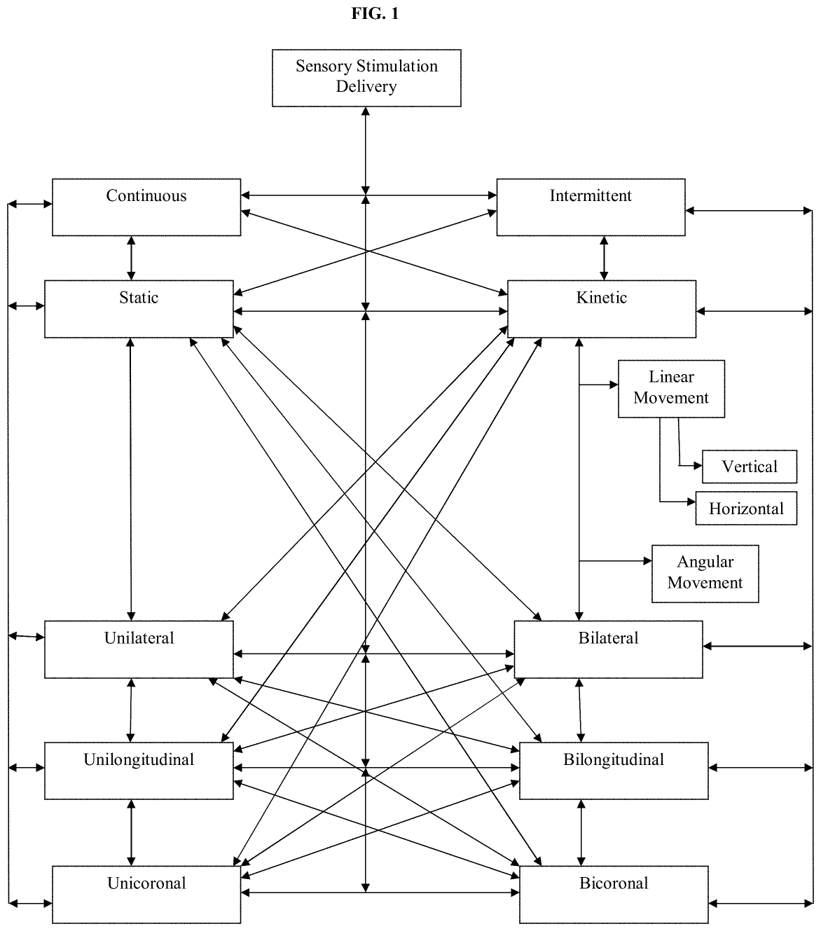

FIG. 2 is a screenshot of the display of a mobile device that is running the mobile application described herein showing one embodiment of the delivery of auditory stimulation as described herein.

FIG. 3 is a screenshot of the display of a mobile device that is running the mobile application described herein showing one embodiment of the delivery of auditory and visual stimulation as described herein, wherein the visual stimulation is bilaterally or bilongitudinally delivered.

FIG. 4 is a screenshot of the display of a mobile device that is running the mobile application described herein showing one embodiment of the intermittent bilateral delivery or bilongitudinal delivery of visual stimulation as described herein.

FIG. 5 is screenshot of the display of a mobile device that is running the mobile application described herein showing one embodiment of the intermittent bilateral delivery or bilongitudinal delivery of visual stimulation as described herein.

### DETAILED DESCRIPTION

### Definitions

As used herein and in the appended claims, the singular forms “a”, “an”, and “the” include plural references unless the context clearly dictates otherwise. Thus, for example, reference to “a sound” is a reference to one or more sounds and equivalents thereof known to those skilled in the art, and so forth.

As used herein, the term “about” means plus or minus 10% of the numerical value of the number with which it is being used. Therefore, about 50% means in the range of 40%-60%.

The terms “acute stress disorder” and “ASD” refer to a psychiatric condition in its broadest sense, as defined in American Psychiatric Association: Diagnostic and Statistical Manual of Mental Disorders, Fifth Edition, Washington, D.C., 2000 (“DSM-5”). The DSM-5 defines “acute stress disorder” as characterized by anxiety, dissociative, and other symptoms occurring within 1 month after exposure to an extreme traumatic stressor. The DSM-5 sets forth a generally accepted standard for diagnosing and categorizing acute stress disorder. The term “acute stress disorder” may be used interchangeably with the term “stress disorder” as used herein.

Anatomical Terms. When referring to humans, the body and its parts are always described using the assumption that the body is standing upright. Portions of the body which are closer to the head end are “superior” (corresponding to the cranial or head end in animals), while those farther away are “inferior.” Objects near the front of the body are referred to as “anterior” (corresponding to ventral in animals); those near the rear of the body are referred to as “posterior” (corresponding to dorsal in animals). Structures near the midline, which is the middle of the body, are called medial and those near the sides of humans are called lateral. Structures that are close to the center of the body are “proximal” or central, while ones more distant are “peripheral” or peripheral. For example, the hands are at the peripheral end of the arms, while the shoulders are at the central ends. Three basic reference planes are used in zoological anatomy. A “sagittal” or “lateral” plane divides the body into left and right portions. A “coronal” plane divides the body into posterior and anterior portions. A “transverse” or “longitudinal” plane divides the body into “superior” and “inferior” portions. A transverse, longitudinal axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal or lateral plane is an X-Z plane, perpendicular to the ground and to the coronal plane, which separates left from right.

The term “anxiety” as used herein refers to a feeling of fear or apprehension about what is to come. Symptoms of general anxiety include: increased heart rate, rapid breathing, restlessness, trouble concentrating, and difficulty falling asleep. An “anxiety attack” is a feeling of overwhelming apprehension, worry, distress, or fear. For many, an anxiety attack builds slowly, and may worsen as a stressful event approaches. Common symptoms of an anxiety attack include: feeling faint or dizzy, shortness of breath, dry mouth, sweating, chills or hot flashes, apprehension and worry; restlessness; distress; and fear.

The term “auditory stimulation” as used herein refers to any stimulation that activates the auditory sense system.

As used herein, the term “auditory system” is used to describe the cells, nerves, and/organs that make up a body sense system in a subject that enables the subject to hear. While the auditory organs vary per species, in humans, the auditory system is divided into two subsystems: the peripheral auditory system, which is comprised of the outer ear, middle ear, and inner ear, and the central auditory system, which extends from the cochlear nucleus up to the primary auditory cortex. The outer ear is comprised of the pinna, the ear canal and the eardrum. The pinna are the parts of the outer ear that appear as folds of cartilage. They surround the ear canal and function as sound wave reflectors and attenuators when the waves hit them. The pinna helps the brain identify the direction from where the sounds originated, for example if they are from near the top of our head (superior side) or from near our feet (inferior side). From the pinna, the sound waves enter a tube-like structure called auditory canal. This canal serves as a sound amplifier. The sound waves travel through the canal and reach the tympanic membrane (eardrum), the canal's end. The middle ear is more complicated. As the sound waves hit the eardrum, the sensory information goes into an air-filled cavity through lever-teletype bones called ossicles. The three ossicles include the hammer (malleus), anvil (incus), and stirrup (stapes). These delicate bones convert the sound vibrations made when the sound waves hit the eardrum into sound vibrations of higher pressure. These transformed vibrations (still in wave form) enter the oval window. Beyond the oval window is the inner ear. This segment of the ear is filled with liquid rather than air, which is why there is a need to convert low pressure sound vibrations to higher pressure ones in the middle ear. The main structure in the inner ear is called the cochlea, where the sensory information in wave form is transformed into the neural form. The cochlear duct contains the organ of Corti, which comprises inner hair cells that turn the vibrations into electric neural signals. Each hair innervates many auditory nerve fibers, and these fibers form the auditory nerve. The auditory nerve (for hearing) combines with the vestibular nerve (for balance), forming cranial nerve VIII or the vestibulocochlear nerve. To reach the central auditory system, once the sound waves are turned into neural signals, they travel through cranial nerve VIII, reaching different anatomical structures where the neural information is further processed. The cochlear nucleus is the first site of neural processing, followed by neural processing by the superior olivary complex located in the pons, and then by the inferior colliculus at the midbrain. The neural information ends up at the thalamus, the relay center of the brain. The auditory information from the thalamus is then passed to the primary auditory cortex of the brain, situated in the temporal lobe. The left posterior superior temporal gyms is responsible for the perception of sound, and in it the primary auditory cortex is the region where the attributes of sound (pitch, rhythm, frequency, etc.) are processed.

The terms “bicoronal” and “unicoronal” as used herein is used to describe the anterior and posterior portions of the body as separated by the coronal or frontal plane.

The terms “bilateral” and “unilateral” as used herein is used to describe the left and right portions of the body as separated by the sagittal or lateral plane.

The terms “bilongitudinal” and “unilongitudinal” as used herein is used to describe the superior and inferior portions of the body as separated by the transverse or longitudinal plane.

The term “cycle” as used herein refers to a series of events that are regularly repeated in the same order.

The terms “disorder” as used herein refer to an impairment of health or a condition of abnormal functioning.

The terms “dissociation” or “dissociative disorder” as used herein refer to the disconnection or lack of connection between things usually associated with each other. Dissociated experiences are not integrated into the usual sense of self, resulting in discontinuities in conscious awareness. In severe forms of dissociation, disconnection occurs in the usually integrated functions of consciousness, memory, identity, or perception. Dissociation may affect a person subjectively in the form of “made” thoughts, feelings, and actions. These are thoughts or emotions seemingly coming out of nowhere, or finding oneself carrying out an action as if it were controlled by a force other than oneself. There are five main ways in which the dissociation of psychological processes changes the way a person experiences living: depersonalization, identity confusion, derealization, amnesia, and identity alteration. The DSM-5 defines dissociation as “a disruption and/or discontinuity in the normal integration of consciousness, memory, identity, emotion, perception, body representation, motor control and behavior” (American Psychiatric Association, 2013, p. 291). The dissociative disorders include: dissociative amnesia (extensive forgetting typically associated with highly aversive events); dissociative fugue (short-lived reversible amnesia for personal identity, involving unplanned travel or “bewildered wandering.” Dissociative fugue is not viewed as a separate disorder but is a feature of some, but not all, cases of dissociative amnesia); depersonalization/derealization disorder (feeling as though one is an outside observer of one's body); and dissociative identity disorder (DID; experiencing two or more distinct identities that recurrently take control over one's behavior) (American Psychiatric Association, 2000). The Structural Clinical Interview for DSM-IV Dissociative Disorders (SCID-D) (Steinberg, 2001) assesses five symptom clusters that encompass key features of the dissociative disorders. These clusters are also found in the DSM-5: depersonalization, derealization, dissociative amnesia, identity confusion, and identity alteration. The Dissociative Experiences Scale (DES) (Bernstein & Putnam, 1986; Carlson & Putnam, 2000; Wright & Loftus, 1999) is the most widely used self-report measure of dissociation.

The term “feeling” as used herein refers to an emotional state, reaction or belief. It can be used to describe a temporary phenomenon that lasts less than 30 seconds, less than 1 minute, less than 5 minutes, less than 10 minutes, less than 20 minutes, less than 30 minutes, less than 40 minutes, less 50 minutes, and less than 60 minutes, inclusive and any value in-between.

As used herein, the term “mobile application” refers to a type of application software designed to run a small, wireless computing device, known as a mobile device, such as a smartphone or a tablet computer. Mobile applications may be designed to work with the hardware configurations the mobile device has, such as a mobile device's display, keyboard, haptics, speakers, and the like, and software integrations, features or capabilities that are programmed or encoded into the mobile device, such as previously existing mobile applications, and the ability to communicate with other electronic devices. A mobile application may also be designed to work with external accessories or attachments publically available or described herein that are configured to communicate with the mobile device. Mobile applications may be also be known colloquially as an “app,” “web app,” “mobile app,” online app,” “smartphone app,” or “tablet app.”

The term “modulate” as used herein means to regulate, alter, adapt, or adjust to a certain measure or proportion. Such modulation may be any change, including an undetectable change.

The term “mood” as used herein refers to a prolonged emotional state, reaction or belief. It can be used to describe a phenomena that is longer lasting than a “feeling” that lasts more than 60 minutes, more than 90 minutes, more than 120 minutes, more than 150 minutes, more than 180 minutes, more than 190 minutes, more than 200 minutes, more than 210 minutes, and more than 220 minutes, inclusive and any value in-between.

As used herein, the term “olfactory stimulation” as used herein is used to describe any stimulation that activates a subject's olfactory system.

The term “olfactory system” is used to describe the cells, nerves and organs and that make up a body sense system in a subject that enables the subject to perceive a smell and to some extent, a taste or flavor. The system consists of the nose and the nasal cavities, which in their upper parts support the olfactory mucous membrane for the perception of smell and in their lower parts act as respiratory passages. The bony framework of the nose is part of the skull, but the outer nose is supported only by bone above; lower down, its shape is kept by cartilaginous plates. The expanded lower part of the side of the nose, the ala, is formed only of skin, both externally and internally, with fibrofatty tissue between the layers. The nasal cavities are separated by a septum covered in its lower two-thirds by a thick, highly vascular mucous membrane composed of columnar ciliated epithelium with masses of acinous glands embedded in it; in its upper part, it is covered by the less vascular but more specialized olfactory membrane. Near the front of the lower part of the septum a slight opening into a short blind tube, which runs upward and backward, may sometimes be found; this is the vestigial remnant of Jacobson's organ. The supporting framework of the septum is made up of ethmoid above, vomer below, and the septal cartilage in front. The outer wall of each nasal cavity is divided into three meatuses by the overhanging turbinated bones. Above the superior turbinated bone is a space between it and the roof known as the recessus sphenoethmoidalis, into the back of which the sphenoidal air sinus opens. Between the superior and middle turbinated bones is the superior meatus, which contains the openings of the posterior ethmoidal air cells, while between the middle and inferior turbinated bones is the middle meatus, which is the largest of the three and contains a rounded elevation, the bulla ethmoidalis. Above and behind this is often an opening for the middle ethmoidal cells; below and in front runs a deep sickle-shaped gutter, the hiatus semilunaris, which communicates above with the frontal air sinus and below with the opening into the antrum of Highmore or maxillary antrum. The inferior meatus is below the inferior turbinated bone, and, when that is lifted, the valvular opening of the nasal duct is seen. The roof of the nose is narrow, and it is here that the olfactory nerves pass in through the cribriform plate. The floor is wider, so that a coronal section through each nasal cavity has roughly the appearance of a right-angled triangle. The pathway of olfactory conduction begins with the olfactory receptors—small, slender nerve cells embedded in large numbers (about 100 million in the rabbit) in the epithelium of the mucous membrane lining the upper part of the nasal cavity. Each olfactory receptor cell emits two processes (projections). One of these is a short peripheral dendrite, which reaches to the surface of the epithelium, where it ends in a knob carrying a number of fine radially placed filaments, the olfactory hairs. The other process is a long and extremely thin axon, the olfactory nerve fiber, which reaches the cranial cavity by passing through one of the openings in the bony roof of the nasal cavity and enters the olfactory bulb of the forebrain. Sensations of smell are experienced when certain chemical substances become dissolved in the thin layer of fluid covering the surface of the mucous membrane and thus come in contact with the olfactory hairs. The receptor cells may differ among themselves in their sensitivities to various odorous substances. In the olfactory bulb, the olfactory nerve fibers end in contact with the antenna-shaped dendrites of the large mitral cells, which represent the second main link in the chain of olfactory conduction. Each mitral cell emits a long axon, many of which enter into the formation of the olfactory tract, a white fiber band extending back from the bulb over the basal surface of the forebrain. The olfactory tract distributes its fibers mainly to the cortex of the pyriform lobe, which constitutes the final cortical receiving area of the olfactory pathway. In humans this region corresponds to the uncus of the hippocampal gyms. A smaller number of fibers of the olfactory tract end in two further olfactory structures; the olfactory tubercle and the medial part of the amygdaloid complex (the latter lies deep to the olfactory cortex). See for example, “Nervous Pathways of Smell.” Encyclopædia Britannica, Encyclopædia Britannica, Inc., https://www.britannica.com/science/olfactory-system/Nervous-pathways-of-smell.

The term “pattern” as used herein refers to combination of qualities, acts or events forming a consistent or characteristic arrangement.

As used herein, the term “perception” and its various grammatical forms refers to an awareness derived from sensory processes while a stimulus is present.

As used herein, the terms “prevention” and “preventing,” when referring to a trauma and stressor-related disorder or symptom, refers to a reduction in the risk or likelihood that a mammalian subject will develop the disorder, symptom, condition, or indicator after utilizing the described invention, or a reduction in the risk or likelihood that a mammalian subject will exhibit a recurrence of the disorder, symptom, condition, or indicator once a subject has utilized the described invention and returned to a normal state (e.g., placed in remission from a targeted disorder).

The term “reduce” and its various grammatical forms as used herein refers to a diminution, a lessening, a decrease, an attenuation or abatement of the degree, intensity, extent, size, amount or number of instances thereof.

As used herein the terms “sense” or “sensory” and its grammatical forms thereof, describes the physiological faculty by which the body of a subject perceives an external stimulus. A subject, such as a human, will perceive an external stimulus through a part of its body, such as: one or more sensory organs, for example, the eyes which provides the ability of sight, the nose and mouth which provides the ability of smell, the ears which provides the ability of hearing, the mouth and tongue which provides the ability of taste, and the skin which provides the ability of touch; or the activation of another part of a subject's body, such as a muscle, gland, or nerve ending.

The term “sequence” as used herein refers to a series of cycles that are regularly repeated in the same order.

The terms “stimulation,” “stimulus,” and “stimuli” as used herein refer to a factor, agent or force that directly influences the activity of a subject, such as by exciting or activating a sensory organ or sensory system, evoking a muscular contraction, or galvanizing a nerve ending. These terms are typically used in conjunction with the terms “sense” or “sensory” to describe exciting or activating the physiological faculty by which the body of a subject perceives a stimulus.

The terms “subject” or “user” or “individual” or “patient” are used interchangeably to refer to a member of an animal species of mammalian origin, including humans, horses, dogs, cats, and the like. According to some embodiments, the terms “subject” or “user” or “individual” or “patient” is also used to refer to “an agent of a subject” wherein the phrase “an agent of a subject” refers to a human authorized to act for, in place of, or on behalf of the subject, for example, if the subject is unable to do so itself. For example, if the subject is an animal, such as a dog, a horse, or other mammal, an agent of the animal may be its owner, trainer, or handler. For example, if the subject is a human, an agent of the human may be his/her/its therapist, assistant, helper, aide, attendant, or caregiver.

The phrase “subject in need thereof” as used herein refers to an individual that has a susceptibility to feeling stress, anxiety or traumatized. A subject may have a susceptibility to feeling trauma if: they have a genetic predisposition to feeling stress, anxiety or traumatized; has exposure to stressful, traumatic, or anxiety-producing environments; is fearful of being exposed to stressful, traumatic, or anxiety-producing environments; has been exposed to an anxiety-producing, traumatic or stressful event; is fearful of recurrence of that anxiety-producing, traumatic or stressful event; and/or any combination of the above.

The term “somatic stimulation” as used herein is used to describe any stimulation that activates a subject's somatosensory system.

The term “somatosensory system” is used to describe the cells, nerves and/organs and that make up a body sense system in a subject that enables the subject to perceive touch, pressure, pain, temperature, position, movement, and vibration, which arise from the muscles, joints, skin, and fascia. The skin is composed of several layers. The very top layer is the epidermis, and the layer below it, known as the dermis, contain very sensitive cells called touch receptors. There are four main types of receptors: mechanoreceptors, thermoreceptors, pain receptors, and proprioceptors that exist in a subject's skin. Each type of receptor is considered a rapidly adapting receptor or a slowly adapting receptor. A receptor is considered rapidly adapting if it responds to a change in stimulus very quickly. Essentially, it can immediately sense when the skin is touching an object and when it stops touching that object. However, rapidly adapting receptors can't sense the continuation and duration of a stimulus touching the skin. These receptors best sense vibrations occurring on or within the skin. A touch receptor is considered slowly adapting if it does not respond to a change in stimulus very quickly. These receptors are very effective at sensing the continuous pressure of an object touching or indenting the skin but are not effective at sensing when the stimulus started or ended. Mechanoreceptors perceive sensations such as pressure, vibrations, and texture. There are four known types of mechanoreceptors that function to perceive indentions and vibrations of the skin: Merkel's disks, Meissner's corpuscles, Ruffini's corpuscles, and Pacinian corpuscles. The most sensitive mechanoreceptors, Merkel's disks and Meissner's corpuscles, are found in the very top layers of the dermis and epidermis and are generally found in non-hairy skin such as the palms, lips, tongue, soles of feet, fingertips, eyelids, and the face. Merkel's disks are slowly adapting receptors, and Meissner's corpuscles are rapidly adapting receptors, which enable the areas of skin in which Merkels disks and Meissner's corpuscles reside to perceive both when the skin is touching something and how long it has been touching something. Located deeper in the dermis and along joints, tendons, and muscles are Ruffini's corpuscles and Pacinian corpuscles. These mechanoreceptors can feel sensations, such as vibrations, traveling down bones and tendons, rotational movement of limbs, and the stretching of skin. Thermoreceptors perceive sensations related to the temperature of objects or the surrounding environment that the skin feels. They are found in the dermis layer of the skin. There are two basic categories of thermoreceptors: hot and cold receptors. Cold receptors start to perceive cold sensations when the surface of the skin drops below 95° F. They are most stimulated when the surface of the skin is at 77° F. and are no longer stimulated when the surface of the skin drops below 41° F. Hot receptors start to perceive hot sensations when the surface of the skin rises above 86° F. and are most stimulated at 113° F.; beyond 113° F., pain receptors take over to avoid damage being done to the skin and underlying tissues. Thermoreceptors are found all over the body, but cold receptors are found in greater density than heat receptors. The highest concentration of thermoreceptors can be found in the face and ears. Pain receptors detect pain or stimuli that can or does cause damage to the skin and other tissues of the body. There are over three million pain receptors throughout the body, found in skin, muscles, bones, blood vessels, and some organs. They can detect pain that is caused by mechanical stimuli (such as a cut or scrape), thermal stimuli (such as a burn), or chemical stimuli (such as poison from an insect sting). Proprioceptors sense the position of the different parts of the body in relation to each other and the surrounding environment. Proprioceptors are found in tendons, muscles, and joint capsules, which allow the cells to detect changes in muscle length and muscle tension. Impulses are carried from receptors via sensory afferents to the dorsal root ganglia, where the cell bodies of the first-order neurons are located. Their axons then travel through the spinal cord either in an ipsilateral or a contralateral fashion. The medulla contains the second-order neurons for fibers carrying touch, position, and vibratory sensations. The fibers are then either conveyed to the thalamus or conveyed to the cerebellum. From the thalamic nucleus, the sensory afferents are projected to the cortical sensory areas, where information is integrated and analyzed (See, for example, Nida Gleveckas-Martens et al. “Somatosensory System Anatomy.” Overview, Gross Anatomy, Microscopic Anatomy, MedScape, 1 Jul. 2019, https://emedieine.medscape.com/article/1948621-overview).

The term “stress” as used herein describes a physical, mental, or emotional state that causes bodily or mental tension. Stresses can be external (such as from the environment, psychological situations, or social situations) or internal (such as from a body illness, mental illness, or medical procedure). Stress can cause the release of catecholamine hormones, such as adrenaline or noradrenaline, which facilitate immediate physical reactions associated with preparation for violent muscular action. These include, without limitation, acceleration of heart and lung activity, paling or flushing, or alternating between both, inhibition of stomach and upper-intestinal action to the point where digestion slows down or stops, a general effect on sphincters of the body, constriction of blood vessels in many parts of the body, liberation of nutrients (particularly fat and glucose) for muscular action, dilation of blood vessels for muscles, inhibition of the lacrimal gland (responsible for tear production) and salivation, dilation of the pupil (mydriasis), relaxation of the bladder, inhibition of erection, auditory exclusion (loss of hearing), tunnel vision (loss of peripheral vision), disinhibition of spinal reflexes, and shaking or trembling. Stress can cause or influence the course of many medical conditions including psychological conditions such as depression and anxiety. Exemplary medical problems resulting from stress can include poor healing, irritable bowel syndrome, high blood pressure, poorly controlled diabetes and many other conditions.

The term “tailoring” and its various grammatical forms as used herein refers to altering, changing, adjusting, amending, modifying, or developing something especially for a particular individual according to his/her/its needs, desires, or preferences, or for a particular purpose according to a goal, aim, intention, objective, aspiration or desire for its use.

The term “therapeutic effect” as used herein refers to a consequence of treatment, the results of which are judged to be desirable and beneficial. A therapeutic effect can include, directly or indirectly, the arrest, reduction, or elimination of a manifestation of a stress disorder, stress, or anxiety. A therapeutic effect can also include, directly or indirectly, the arrest, reduction or elimination of the progression of a manifestation of a stress disorder, stress, or anxiety.

As used herein the term “treating” includes abrogating, substantially inhibiting, slowing or reversing the progression of a condition, substantially ameliorating symptoms of a condition, or substantially preventing the appearance of symptoms of a condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). Treatment also includes eliciting a significant response, whether detectable or undetectable, without excessive levels of side effects.

The term “vestibular stimulation” as used herein refers to any stimulation that activates the vestibular system.

As used herein “vestibular system” is used to describe the cells, organs, and nerves that make up a body sense system in a subject that enables the subject to feel motion and spatial orientation. The vestibular system comprises several structures and tracts. The main components of the system are found in the inner ear in a system of interconnected compartments called the vestibular labyrinth. The vestibular labyrinth is made up of the semicircular canals and the otolith organs, and contains receptors for vestibular sensations. These receptors send vestibular information via the vestibulocochlear nerve to the cerebellum and to nuclei in the brainstem called the vestibular nuclei. The vestibular nuclei then pass the information on to a variety of targets, ranging from the muscles of the eye to the cerebral cortex. Vestibular sensations begin in the inner ear in the vestibular labyrinth, a series of interconnected chambers that are continuous with the cochlea. The semicircular canals of the vestibular labyrinth consist of three tubes, positioned approximately at right angles to one another that are each situated in a plane in which the head can rotate. This design allows each of the canals to detect one of the following head movements: nodding up and down, shaking side to side, or tilting left and right. These movements of the head around an axis are referred to as rotational acceleration, and can be contrasted with linear acceleration, which involves movement forward or backward. The semicircular canals are filled with a fluid called endolymph, which is similar in composition to the intracellular fluid found within neurons. When the head is rotated, it causes the movement of endolymph through the canal that corresponds to the plane of the movement. The endolymph in that semicircular canal flows into an expansion of the canal called the ampulla. Within the ampulla is a sensory organ called the crista ampullaris that contains hair cells, the sensory receptors of the vestibular system. Hair cells contain small “hairs” called stereocilia extending from the top of each cell. Hair cell stereocilia have fine fibers, known as tip links, that run between their tips; tip links are also attached to ion channels. When the stereocilia of hair cells are moved, the tip links pull associated ion channels open for a fraction of a millisecond. This is long enough to allow ions to rush through the ion channels to cause depolarization of the hair cells. Depolarization of the hair cells leads to a release of neurotransmitters and the stimulation of the vestibulocochlear nerve. The hair cells associated with the semicircular canals extend out of the crista ampullaris into a gelatinous substance called the cupula, which separates hair cells from the endolymph. When the endolymph flows into the ampulla, however, it causes the distortion of the cupula, which leads to movement of hair cells. This prompts stimulation of the vestibulocochlear nerve, which transmits the information about head movement to the vestibular nuclei in the brainstem as well as to the cerebellum. The vestibular system uses two other organs, known as the otolith organs, to detect linear acceleration, gravitational forces, and tilting movements. There are two otolith organs in the vestibular labyrinth: the utricle and the saccule. The utricle is specialized to detect movement in the horizontal plane, while the saccule detects movement in the vertical plane. The process of sensation in the otolith organs bears some similarity to the process in the semicircular canals, but there are also some distinct differences. Like the semicircular canals, the otolith organs also contain a sensory organ where hair cells can be found; in this case, however, it is called the macula. As in the semicircular canals, there is a gelatinous layer above the hair cells; in the otolith organs, however, there is another fibrous structure called the otolithic membrane above the gelatinous layer. The otolithic membrane has small crystals of calcium carbonate called otoconia embedded within it. These crystals make the otolithic membrane heavier than the rest of the structure; when linear acceleration occurs, it causes the otolithic membrane to shift relative to the macula, which leads to the displacement of hair cells and thus the release of neurotransmitters from these cells. The structure of the otolith organs makes them especially sensitive to movements like linear acceleration and head tilts. The vestibular system uses this information about movement obtained via the semicircular canals and otolith organs to maintain balance, stability, and posture. (See, Khan S, Chang R (2013). Anatomy of the vestibular system: A review NeuroRehabilitation, 32 (3), 437-443). As used herein, the above description is referred to in this application as the central vestibular sensory system, wherein stimulation thereof can be through central movements, such as movements of the subject's head. The somatic sensory system and the vestibular sensory system are closely related, where proprioreceptors in the somatic sensory system that are found in a subject's skin also act in the vestibular sensory system to send messages to the brain to maintain balance, stability, and posture. The above description is referred to in this application as the peripheral vestibular sensory system, wherein stimulation thereof can be through peripheral movements, such as movements of the subject's feet.

The term “visual stimulation” as used herein is used to describe any stimulation that activates a subject's visual system. As used herein, the term “visual system” is used to describe the cells, nerves, and organs that make up a body sense system in a subject that enables the subject to see. Essentially, the visual system begins with the eye where visual information is carried by the axons of the retinal ganglion cells to the midbrain and diencephalon. The visual system is complex as much of visual processing occurs outside the brain within the retina of the eye. Images are formed in the eye by refraction, the bending of light rays that can occur when they travel from one transparent medium to another. Lightwaves enter the eye through the cornea, refract, go through the pupil and reaches the retina. The pigmentation of the eye is provided by the iris; the white part of the eye is called sclera and it forms the eyeball; extraocular muscles move the eye and the eye's orbit. The axons exit the retina through the optic nerve in the back of the eye. Image formation in the eye happens in the retina according to basic optical properties. The retina consists of several layers of cells, some of which act as receptors. The light wave information propagates through these layers as follows: photoreceptors respond to light and influence the membrane potential of the bipolar cells, and ganglion cells fire action potentials which propagate through the optic nerve to the rest of the brain for visual processing. (see for example, Valentin Dragoi. “Visual Processing: Eye and Retina (Section 2, Chapter 14) Neuroscience Online: An Electronic Textbook for the Neurosciences: Department of Neurobiology and Anatomy—The University of Texas Medical School at Houston.” Neuroscience Online: An Electronic Textbook for the Neurosciences Department of Neurobiology and Anatomy—The University of Texas Medical School at Houston, University of Texas, https://nba.uth.tmc.edu/neuroscience/m/s2/chapter14.html; see also Hon-Vu Quang Duong et al. “Visual System Anatomy.” Visual System Anatomy, MedScape, 1 Jul. 2019, https://emedicine.medscape.com/article/1948576-overview).

The term “wellness” as used herein refers to a good or satisfactory condition of existence; a state characterized by physical and mental health; a sense of well-being.

### Methods of Use

The described mobile application can improve the wellness of a subject. According to one aspect, the described invention provides a method for improving wellness of a subject comprising providing the subject the wellness mobile application described herein and allowing the subject to interact with the mobile application.

The mobile application described herein leverages existing mobile “smart phones” and “tablet computers.” By way of example only, and not by way of limitation, such smartphones include Apple's iPhone series, Google's Droid and Nexus One series, Palm's Pre series, Samsung's Galaxy series, LG's G/V/K/Tribute/Stylo/Pheonix/Optimus/X/Nexus/and Fiesta series, and RIM's Blackberry series of smart phones. By way of example only, and not by way of limitation, such tablets include Apple's iPad series, Google's Pixel series, Samsung's Galaxy Tab series, Asus ZenPad series, Microsoft's Surface series, LG's G pad series, Amazon's Fire series, Lenovo's Tab series, and Huawei's MediaPad series of tablets. For description purposes, it is understood that each of a smartphone and a tablet comprises a touchscreen display, a keyboard, speakers, at least one camera, a lightbulb, a port for charging the device, and the ability to connect to or “communicate” with other external accessory devices digitally, such as by Bluetooth, or electronically, such as by cable connections. Phone accessory devices are referred herein as “accessories” or “attachments.”

According to some embodiments, the described invention provides a method for improving the perception of wellness by a subject comprising:
Providing a wellness improving mobile application, wherein the mobile application is designed to deliver at least one sensory stimulation;
Delivering the at least one sensory stimulation designed to deliver the sensory stimulation to the subject; and
Allowing the subject to interact with the mobile application, such interaction improving a perception of wellness by the subject.

Improvement of the perception of wellness by the subject includes, for example: improving a perception of wellness by the subject by: reducing and/or alleviating a level of one or more negative feelings felt by a subject; invoking, increasing, and/or maintaining a level of one or more positive feelings felt by a subject; reducing and/or alleviating a negative mood felt by a subject; invoking, increasing, and/or maintaining a positive mood felt by a subject; inducing the prevalence or occurrence of one or more desirable physiological responses, a reduction of the prevalence or occurrence of one or more undesirable physiological responses; or any combination of the above.

According to some embodiments, the methods of the present invention may be effective to improve the perception of wellness by a subject by at least 1%, by at least 5%, by at least 10%, by at least 15%, by at least 20%, by at least 25%, by at least 30%, by at least 35%, by at least 40%, by at least 45%, by at least 50%, by at least 60%, by at least 70%, by at least 80%, by at least 90%, by at least 100% when compared to a subject not receiving a described composition thereof (“an untreated control”).

According to some embodiments, the methods of the present invention may be effective for a subject as defined herein, wherein a “subject” refers to a member of an animal of species of mammalian origin, including humans, horses, dogs, and cats. According to some embodiments, the subject is a human. According to some embodiments, the subject is a horse. According to some embodiments, the subject is a horse that participates in competitions or sporting activities, such as, racing, driving, conformation, showmanship, vaulting, showjumping, trail riding, polo, rodeo events, endurance riding, dressage, western dressage, hunt seat, jumping, eventing, reining, western pleasure, parade, drill team, trick riding, mounted games, and the like. According to some embodiments, the subject is a horse that serves, assists, is used, ridden, or trained for use in various operations, such as for mounted police patrol, border patrol, crowd control, mounted warfare, warfare pulling, cavalry, and the like. According to some embodiments, the subject is a dog. According to some embodiments, the subject is a dog that participates in competitions or sporting activities, such as, agility, bikejoring, canicross, caniteering, carting, competition obedience, conformation showing, catchball, disc dog, dock jumping, dog hiking, pack hiking, earthdog trials, field trials, flyball, French ring sport, greyhound racing, hare coursing, herding or stock dog, hunting, hound trailing, junior showmanship, lure coursing, mushing, dog mushing, musical canine freestyle, canine dressage, heelwork to music, nosework, obedience training, protection sports, rally obedience retrieving trials, scootering, sheepdog trials, sighthound disc sport, sighthound racing, racing, coursing, lure coursing, skijoring, sled dog racing, dog surfing, terrier racing, tracking trials, treibball, waterwork, water-rescue, and weight pulling. According to some embodiments, the subject is a dog that serves, assists, is used, or trained for use in various operations such as emotional comfort, mental disability, detection for explosives or other illegal substances, tracking, search, rescue, wilderness guide, sight guide, disability assistance, scouting, patrolling, messenger, mine, casualty (e.g., after natural disaster or terror attack), tunnel, hearing, diabetic alert, mobility assistance, seizure response, autism support, psychiatric service, and the like.

### Emotional, Mental and Physiological Responses

According to some embodiments, improving the perception of wellness by a subject comprises reducing and/or alleviating the level of one or more negative feelings felt by a subject. As used herein, the term “negative feelings” means feelings that invoke, increase, or maintain feelings of agitation, disruption, disturbance, overexcitement, trouble, stress, anxiety, clamor, turbulence, burden, fear, fearfulness, nervousness, strain, tension, trauma, worry, affliction, alarm, apprehension, apprehensiveness, disquiet, disquietude, distention, dread, impatience, restlessness, tautness, tenseness, tensity, irritation, irritability, tightness, trepidation, nervous tension, overextension, overwhelm, urgency, upheaval, discomposure, commotion, turmoil, discord, dislike, angst, jitters, misery, misgiving, panic, suffering, uncertainty, unease, distress, foreboding, fretfulness, worriment, frustration, solicitude, derealization, and/or depersonalization, or any combination thereof.

The method for reducing and/or alleviating one or more “negative feelings” felt by a subject comprises providing the mobile application described herein and allowing the subject to interact with the mobile application. According to some embodiments, the methods of the present invention may be effective for a subject to experience, subjectively, objectively, or both, a reduced and/or alleviated level of negative feelings felt by a subject by at least 1%, by at least 5%, by at least 10%, by at least 15%, by at least 20%, by at least 25%, by at least 30%, by at least 35%, by at least 40%, by at least 45%, by at least 50%, by at least 60%, by at least 70%, by at least 80%, by at least 90%, by at least 100% when compared to a an untreated control.

According to some embodiments, improving the perception of wellness by a subject comprises invoking, increasing, and/or maintaining the level of one or more positive feelings felt by a subject. As used herein the term “positive feelings” means feelings that evoke, increase, or maintain feelings of relief, peace, calm, serenity, tranquility, relaxation, security, safety, satisfaction, comfort, well-being, at ease, optimism, pleasant, bliss, cheerfulness, composure, patience, stillness, placidity, placidness, quietness, quietude, contentment, untroubledness, rest, restfulness, harmony, order, accord, unanimity, affinity, compatibility, and/or amicability, or any combination thereof of a subject. As used herein the term “positive feelings” further means feelings that reduce, or alleviate “negative feelings” as described herein.

The method for evoking, increasing, and/or maintaining one or more “positive feelings” felt by a subject comprises providing the mobile application described herein and allowing the subject to interact with the mobile application. According to some embodiments, the methods of the present invention may be effective for a subject to experience, subjectively, objectively, or both, an increased and/or maintained level of positive feelings felt by a subject by at least 1%, by at least 5%, by at least 10%, by at least 15%, by at least 20%, by at least 25%, by at least 30%, by at least 35%, by at least 40%, by at least 45%, by at least 50%, by at least 60%, by at least 70%, by at least 80%, by at least 90%, by at least 100% when compared to an untreated control.

According to some embodiments, improving the perception of wellness by a subject comprises reducing and/or alleviating a “negative mood” felt by a subject. As used herein, the term “negative mood” means a state of mind that invokes, increases, or maintains one or more feelings of agitation, disruption, disturbance, overexcitement, trouble, stress, anxiety, clamor, turbulence, burden, fear, fearfulness, nervousness, strain, tension, trauma, worry, affliction, alarm, apprehension, apprehensiveness, disquiet, disquietude, distention, dread, impatience, restlessness, tautness, tenseness, tensity, irritation, irritability, tightness, trepidation, nervous tension, overextension, overwhelm, urgency, upheaval, discomposure, commotion, turmoil, discord, dislike, angst, jitters, misery, misgiving, panic, suffering, uncertainty, unease, distress, foreboding, fretfulness, worriment, frustration, solicitude, derealization, and/or depersonalization, or any combination thereof of a subject.

The method for reducing and/or alleviating a “negative mood” felt by a subject comprises providing the mobile application described herein and allowing the subject to interact with the mobile application. According to some embodiments, the methods of the present invention may be effective for a subject to experience, subjectively, objectively, or both, a reduced and/or alleviated level of a “negative mood” felt by a subject for at least 30 seconds, for at least 1 minute, for at least 2 minute, for at least 3 minutes, for at least 4 minutes, for at least 5 minutes, for at least 6 minutes, for at least 7 minutes, for at least 8 minutes, for at least 9 minutes, for at least 10 minutes, for at least 20 minutes, for at least 30 minutes, for at least 40 minutes, for at least 50 minutes, for at least 1 hour, for at least 1.5 hours, for at least 2 hours, for at least 2.5 hours, and for at least 3 hours after the subject initially interacts with the mobile application.

According to some embodiments, improving the perception of wellness by a subject comprises invoking, increasing, and/or maintaining a “positive mood” felt by a subject. As used herein the term “positive mood” means a state of mind that evokes, increases, or maintain feelings of one or more of relief, peace, calm, serenity, tranquility, relaxation, security, safety, satisfaction, comfort, well-being, at ease, optimism, pleasant, bliss, cheerfulness, composure, patience, stillness, placidity, placidness, quietness, quietude, contentment, untroubledness, rest, restfulness, harmony, order, accord, unanimity, affinity, compatibility, and/or amicability, or any combination thereof of a subject.

The method for evoking, increasing, and/or maintaining a “positive mood” felt by a subject comprises providing the mobile application described herein and allowing the subject to interact with the mobile application. According to some embodiments, the methods of the present invention may be effective for a subject to experience, subjectively, objectively, or both, an increase, and/or maintenance of a positive mood felt by a subject for at least 30 seconds, for at least 1 minute, for at least 2 minute, for at least 3 minutes, for at least 4 minutes, for at least 5 minutes, for at least 6 minutes, for at least 7 minutes, for at least 8 minutes, for at least 9 minutes, for at least 10 minutes, for at least 20 minutes, for at least 30 minutes, for at least 40 minutes, for at least 50 minutes, for at least 1 hour, for at least 1.5 hours, for at least 2 hours, for at least 2.5 hours, and for at least 3 hours after the subject initially interacts with the mobile application, inclusive and any value in between.

According to some embodiments, improving the perception of wellness by a subject comprises inducing desirable physiological responses in a subject. As used herein the term “desirable physiological responses” means the prevalence or occurrence of one or more of a transient parethesia (meaning a sensation of tickling or tingling), normalized blood pressure (systolic being less than about 120 mm Hg and diastolic being less than about 80 mm Hg for an adult), piloerection (goosebumps), mydriasis (dilated pupils), normalized heart rate (about 60 beats per minute for an adult), and/or a normalized respiration rate (from about 12 to about 20 breaths per minute for an adult), or any combination thereof.

The method for inducing desirable physiological responses felt by a subject comprises providing the mobile application described herein and allowing the subject to interact with the mobile application. According to some embodiments, the methods of the present invention may be effective for a subject to experience, subjectively, objectively, or both, a desirable physiological response for at least 30 seconds, for at least 1 minute, for at least 2 minute, for at least 3 minutes, for at least 4 minutes, for at least 5 minutes, for at least 6 minutes, for at least 7 minutes, for at least 8 minutes, for at least 9 minutes, for at least 10 minutes, for at least 20 minutes, for at least 30 minutes, for at least 40 minutes, for at least 50 minutes, for at least 1 hour, for at least 1.5 hours, for at least 2 hours, for at least 2.5 hours, and for at least 3 hours after the subject initially interacts with the mobile application, inclusive and any value in between.

According to some embodiments, improving the perception of wellness by a subject comprises reducing undesirable physiological responses in a subject. As used herein the term “undesirable physiological responses” means the prevalence or occurrence of one or more of high blood pressure (systolic being greater than about 120 mm Hg and diastolic being greater than about 80 mm Hg in an adult; accelerated heart rate (greater than about 60 beats per minute in an adult), palpitations, pounding heart; accelerated respiration rate (greater than from about 12 breaths per minute to about 20 breaths per minute), sensations of shortness of breath or smothering; nausea or abdominal distress, feeling dizzy, unsteady, light-headed or faint; chills or heat sensations; sweating; trembling or shaking; sensation of tightness in the throat or of choking; chest pain or discomfort; muscle tension; fatigue; insomnia; dry mouth; and headache or migraine.

The method for reducing undesirable physiological responses felt by a subject comprises providing the mobile application described herein and allowing the subject to interact with the mobile application. According to some embodiments, the methods of the present invention may be effective for a subject to experience, subjectively, objectively, or both, one or more reduced undesirable physiological responses for at least 30 seconds, for at least 1 minute, for at least 2 minute, for at least 3 minutes, for at least 4 minutes, for at least 5 minutes, for at least 6 minutes, for at least 7 minutes, for at least 8 minutes, for at least 9 minutes, for at least 10 minutes, for at least 20 minutes, for at least 30 minutes, for at least 40 minutes, for at least 50 minutes, for at least 1 hour, for at least 1.5 hours, for at least 2 hours, for at least 2.5 hours, and for at least 3 hours after the subject initially interacts with the mobile application, inclusive and any value in between.

According to some embodiments, improving the perception of wellness by a subject comprises increasing a perception of the ability to focus, concentrate, fixate, direct attention towards, focus attention towards, fix attention to, or increase the ability of the foregoing thereof, or both. For example, the present method may be effective to increase the subject's ability to learn, study, train, be trained, and the like.

According to some embodiments, improving the perception of wellness by a subject comprises decreasing the perception of distraction, nonfocusedness, confusion, bewilderment, disorientation, demoralization, befuddlement, perplexity, perturbation, or decreasing the ability of the foregoing thereof, or both. For example, the present method may be effective to increase the subject's ability to learn, study, train, be trained, and the like.

According to some embodiments, improving the perception of wellness by the subject comprises providing a subject with the mobile application described herein, and allowing the subject to interact with the described mobile application, and the subject experiencing, subjectively, objectively, or both, an emotional, mental, and/or physiological response that results in: a cessation, reduction and/or alleviation of one or more negative feelings; an invocation, increase, and/or maintenance in a level of one or more positive feelings; cessation, reduction and/or alleviation of a negative mood; an invocation, increase, and/or maintenance of a positive mood; an inducement of one or more desirable physiological responses; a cessation or reduction of one or more undesirable physiological responses; or any combination of the above.

For example, the subject may experience an emotional, mental, and/or physiological response that results in a cessation, reduction and/or alleviation in the level of one or more negative feelings. In another example, the subject may experience an emotional, mental, and/or physiological response that results in a cessation, reduction and/or alleviation in the level of one or more negative feelings and an invocation, increase, and/or maintenance in the level of one or more positive feelings. In another example, the subject may experience an emotional, mental, and/or physiological response that results in a cessation, reduction and/or alleviation in the level of one or more negative feelings; an invocation, increase, and/or maintenance in the level of one or more positive feelings; and a cessation, reduction and/or alleviation of a negative mood. In another example, the subject may experience an emotional, mental, and/or physiological response that results in a cessation, reduction and/or alleviation in the level of one or more negative feelings; an invocation, increase, and/or maintenance in the level of one or more positive feelings; a cessation, reduction and/or alleviation of a negative mood; and an invocation, increase, and/or maintenance of a positive mood. In another example, the subject may experience an emotional, mental, and/or physiological response that results in a cessation, reduction and/or alleviation in the level of one or more negative feelings; an invocation, increase, and/or maintenance in the level of one or more positive feelings; a cessation, reduction and/or alleviation of a negative mood; an invocation, increase, and/or maintenance of a positive mood; and an inducement of the prevalence or occurrence of one or more desirable physiological responses. In another example, the subject may experience an emotional, mental, and/or physiological response that results in a cessation, reduction and/or alleviation in the level of one or more negative feelings; an invocation, increase, and/or maintenance in the level of one or more positive feelings; a cessation, reduction and/or alleviation of a negative mood; an invocation, increase, and/or maintenance of a positive mood; an inducement of the prevalence or occurrence of one or more desirable physiological responses; and a cessation, reduction of the prevalence or occurrence of one or more undesirable physiological responses.

### Times of Stress or Anxiety

According to some embodiments, the methods of the present invention may be effective in a variety of situations, such as during times of stress or anxiety, prior to times of stress or anxiety, and after times of stress or anxiety. The term “times of stress or anxiety” as used herein is used to describe stressful or anxiety-provoking environmental, physical or emotional cues. Environmental cues can include for example, loud noises, noxious odors, bright lights, crowds, fast-paced movement, travel, a testing environment, a work environment, the illness or passing of a subject's loved one, and the like. Physical cues can include, for example, using and withdrawing from addictive substances, such as alcohol, caffeine, and nicotine; a genetic predisposition and/or a family history of mental disorders; and injury or illness. Emotional cues can include for example, conflict, or the feeling of guilt and/or failure stemming from a subject's personal relationships, job, school, or finances; and the like. According to some embodiments, the methods of the present invention may be effective for a subject to experience, subjectively, objectively, or both, in a variety of situations, such as in times of stress or anxiety, prior to times of stress or anxiety, and after times of stress or anxiety a reduced and/or alleviated level of stress or anxiety by at least 1%, by at least 5%, by at least 10%, by at least 15%, by at least 20%, by at least 25%, by at least 30%, by at least 35%, by at least 40%, by at least 45%, by at least 50%, by at least 60%, by at least 70%, by at least 80%, by at least 90%, by at least 100% when compared to an untreated control.

According to some embodiments, the step of providing a subject with the mobile application described herein further comprises providing the subject with the mobile application during times of stress or anxiety, prior to times of stress or anxiety, and after times of stress or anxiety, wherein the subject thereby experiences: a cessation, reduction and/or alleviation in the level of one or more negative feelings; an invocation, increase, and/or maintenance in the level of one or more positive feelings; cessation, reduction and/or alleviation of a negative mood; an invocation, increase, and/or maintenance of a positive mood; an inducement of the prevalence or occurrence of one or more desirable physiological responses, an cessation, reduction of the prevalence or occurrence of one or more undesirable physiological responses; or any combination of the above. According to some embodiments, the described invention provides a method that is effective to refocus a subject's attention or concentration during times of stress or anxiety, prior to times of stress or anxiety, and after times of stress or anxiety. According to some embodiments, the methods of the present invention may be effective as determined subjectively, objectively, or both, to refocus a subject's attention or concentration by at least 1%, by at least 5%, by at least 10%, by at least 15%, by at least 20%, by at least 25%, by at least 30%, by at least 35%, by at least 40%, by at least 45%, by at least 50%, by at least 60%, by at least 70%, by at least 80%, by at least 90%, by at least 100% when compared to an untreated control.

According to some embodiments, the step of providing a subject with the mobile application described herein further comprises providing the subject with the mobile application to refocus a subject's attention or concentration wherein the subject thereafter experiences: a cessation, reduction and/or alleviation in the level of one or more negative feelings; an invocation, increase, and/or maintenance in the level of one or more positive feelings; cessation, reduction and/or alleviation of a negative mood; an invocation, increase, and/or maintenance of a positive mood; an inducement of the prevalence or occurrence of one or more desirable physiological responses, a cessation, reduction of the prevalence or occurrence of one or more undesirable physiological responses; or any combination of the above. According to some embodiments, the step of providing a subject with the mobile application described herein further comprises providing the subject with the mobile application during times of stress or anxiety, prior to times of stress or anxiety, and after times of stress or anxiety to refocus a subject's attention or concentration so that the subject experiences: a cessation, reduction and/or alleviation in the level of one or more negative feelings; an invocation, increase, and/or maintenance in the level of one or more positive feelings; cessation, reduction and/or alleviation of a negative mood; an invocation, increase, and/or maintenance of a positive mood; an inducement of the prevalence or occurrence of one or more desirable physiological responses, a cessation, reduction of the prevalence or occurrence of one or more undesirable physiological responses; or any combination of the above.

According to some embodiments, the method of the present invention is effective, subjectively, objectively, or both, to increase a subject's ability to cope in a variety of situations, such as in times of stress or anxiety situations, prior to times of stress or anxiety, and after times of stress or anxiety. As used herein, the term “ability to cope” describes a subject's ability to manage, handle, survive, get through, and/or get by in difficult situations, for example in times of stress or anxiety, prior to times of stress or anxiety, and after times of stress or anxiety.

According to some embodiments, the methods of the present invention may be effective subjectively, objectively, or both to increase a subject's ability to cope by at least 1%, by at least 5%, by at least 10%, by at least 15%, by at least 20%, by at least 25%, by at least 30%, by at least 35%, by at least 40%, by at least 45%, by at least 50%, by at least 60%, by at least 70%, by at least 80%, by at least 90%, by at least 100% when compared to an untreated control.

According to some embodiments, the step of providing a subject with the mobile application described herein further comprises providing the subject with the mobile application to increase a subject's ability to cope, as a result of which the subject experiences: a cessation, reduction and/or alleviation in the level of one or more negative feelings; an invocation, increase, and/or maintenance in the level of one or more positive feelings; cessation, reduction and/or alleviation of a negative mood; an invocation, increase, and/or maintenance of a positive mood; an inducement of the prevalence or occurrence of one or more desirable physiological responses, a cessation, reduction of the prevalence or occurrence of one or more undesirable physiological responses; or any combination of the above. According to some embodiments, the step of providing a subject with the mobile application described herein further comprises providing the subject with the mobile application to increase a subject's ability to cope during times of stress or anxiety, prior to times of stress or anxiety, and after times of stress or anxiety, wherein as a result, the subject experiences: a cessation, reduction and/or alleviation in the level of one or more negative feelings; an invocation, increase, and/or maintenance in the level of one or more positive feelings; cessation, reduction and/or alleviation of a negative mood; an invocation, increase, and/or maintenance of a positive mood; an inducement of the prevalence or occurrence of one or more desirable physiological responses, a cessation, reduction of the prevalence or occurrence of one or more undesirable physiological responses; or any combination of the above.

Sensory Stimulation

According to some embodiments, the methods of the present invention may be effective to improve the wellness of a subject by providing a mobile application that delivers at least one sensory stimulation, such as, visual stimulation, auditory stimulation, somatic stimulation, olfactory stimulation, and/or vestibular stimulation. According to some embodiments, the methods of the present invention may be effective to reduce and/or alleviate the level of negative feelings of a subject by providing a mobile application that delivers at least one sensory stimulation including a visual stimulation, an auditory stimulation, a somatic stimulation, an olfactory stimulation, and/or a vestibular stimulation. According to some embodiments, the methods of the present invention may be effective to invoke, increase, and/or maintain the level of positive feelings of the subject by providing a mobile application that delivers at least one sensory stimulation, such as, visual stimulation, auditory stimulation, somatic stimulation, olfactory stimulation, and/or vestibular stimulation. According to some embodiments, the methods of the present invention may be effective to reduce and/or alleviate a subject's negative mood by providing a mobile application that delivers at least one sensory stimulation, including visual stimulation, auditory stimulation, somatic stimulation, olfactory stimulation, and/or vestibular stimulation. According to some embodiments, the methods of the present invention may be effective to invoke, increase, and/or maintain a subject's positive mood by providing a mobile application that delivers at least one sensory stimulation including visual stimulation, auditory stimulation, somatic stimulation, olfactory stimulation, and/or vestibular stimulation. According to some embodiments, the methods of the present invention may be effective to induce the prevalence or occurrence of desirable physiological responses in a subject by providing a mobile application that delivers at least one sensory stimulation including visual stimulation, auditory stimulation, somatic stimulation, olfactory stimulation, and/or vestibular stimulation. According to some embodiments, the methods of the present invention may be effective to reduce the prevalence or occurrence of undesirable physiological responses in a subject by providing a mobile application that delivers at least one sensory stimulation including visual stimulation, auditory stimulation, somatic stimulation, olfactory stimulation, and/or vestibular stimulation. According to some embodiments, the methods of the present invention may be effective to improve the wellness of a subject during times of stress, prior to times of stress, and after times of stress by providing a mobile application that delivers at least one sensory stimulation including visual stimulation, auditory stimulation, somatic stimulation, olfactory stimulation, and/or vestibular stimulation. According to some embodiments, the methods of the present invention may be effective to increase the subject's ability to cope by providing a mobile application that delivers at least one sensory stimulation including visual stimulation, auditory stimulation, somatic stimulation, olfactory stimulation, and/or vestibular stimulation. According to some embodiments, the methods of the present invention may be effective to refocus a subject's attention or concentration by providing a mobile application that delivers at least one sensory stimulation including visual stimulation, auditory stimulation, somatic stimulation, olfactory stimulation, and/or vestibular stimulation. According to some embodiments, the methods of the present invention may be effective to increase the subject's ability to cope during times of stress, prior to times of stress, and after times of stress by providing a mobile application that delivers at least one sensory stimulation including visual stimulation, auditory stimulation, somatic stimulation, olfactory stimulation, and/or vestibular stimulation. According to some embodiments, the methods of the present invention may be effective to refocus a subject's attention or concentration during times of stress, prior to times of stress, and after times of stress by providing a mobile application that delivers at least one sensory stimulation including visual stimulation, auditory stimulation, somatic stimulation, olfactory stimulation, and/or vestibular stimulation.

According to some embodiments, the step of providing a mobile application that delivers sensory stimulation comprises the delivery of one or more of sensory stimulation comprising visual stimulation, auditory stimulation, somatic stimulation, olfactory stimulation, and/or vestibular stimulation. According to some embodiments, the mobile application of the present invention delivers a visual stimulation. According to some embodiments, the mobile application of the present invention delivers a visual stimulation, and an auditory stimulation. According to some embodiments, the mobile application of the present invention delivers a visual stimulation, an auditory stimulation, and a somatic stimulation. According to some embodiments, the mobile application of the present invention delivers a visual stimulation, an auditory stimulation, a somatic stimulation, and an olfactory stimulation. According to some embodiments, the mobile application of the present invention delivers a visual stimulation, an auditory stimulation, a somatic stimulation, an olfactory stimulation, and a vestibular stimulation.

According to some embodiments, visual stimulation comprises the emittance or transmittance of light, graphics, images, videos, text, and the like, and any combination thereof. Light may be emitted to be viewed as any form such as colored, white, patterned, still, in shapes, amorphous, intensity, and the like, or any combination thereof. Images may be transmitted to be viewed as any form, including photographic, designed such as by graphic design or hand drawing, still, or in graphic interchange format (“GIF”), and the like or a combination thereof. Videos may be transmitted to be viewed in any form such as in animation form, live action form, and the like or a combination thereof. Text may be transmitted to be viewed in any form, including any variation of font, size, formatting, style, language, and the like, and in any combination thereof. Visual stimulation can be emitted by or transmitted from the mobile phone screen, the mobile phone light bulb, or any suitable accessory that can communicate with the mobile application.

According to some embodiments, the visual stimulation can be delivered at one or more wavelengths, or within a range of wavelengths. For example, the visual stimulation can be set to be delivered at wavelengths in any range, e.g., within an upper limit of about 740 nm and a lower limit of about 380 nm, inclusive. According to some embodiments, the stimulation can be set to be delivered at a wavelength of about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, or about 740 nm, inclusive and any value in between. According to some embodiments, the stimulation can be set to be delivered at wavelengths ranging from about 380 nm to about 450 nm, inclusive. According to some embodiments, the visual stimulation can be set to be delivered at wavelengths ranging from about 450 nm to about 485 nm, inclusive. According to some embodiments, the visual stimulation can be set to be delivered at wavelengths from about 485 nm to about 500 nm, inclusive. According to some embodiments, the visual stimulation can be set to be delivered at wavelengths from about 500 nm to about 565 nm, inclusive. According to some embodiments, the visual stimulation can be set to be delivered at wavelengths from about 565 nm to about 590 nm, inclusive. According to some embodiments, the visual stimulation can be set to be delivered at wavelengths from about 590 nm to about 625 nm, inclusive. According to some embodiments, the visual stimulation can be set to be delivered at wavelengths from about 625 nm to about 740 nm, inclusive. Those of skill in the art will recognize that each of the above wavelengths can be used as a boundary for a range of wavelengths.

According to some embodiments, the auditory stimulation can include the emittance or transmittance of a musical sound, a non-musical sound, speech, and the like and any combination thereof. According to some embodiments, any type of musical sound can be emitted to be heard in any form, such as, a note or pitch, or a plurality thereof as vocal, instrumental, electronic, and the like. Any type of non-musical sound can be emitted to be heard in any form, such as, a pitch, a frequency, a vibration, a plurality thereof such as, white noise, brown noise, pink noise, green noise, and sounds found in the environment, such as the sound of a brook, ocean waves crashing on a beach, raindrops falling against a window, and the like. According to some embodiments, any type of speech may be emitted to be heard in any form, such as, a whisper, purr, murmur, hum, words, songs, stories, and the like. Auditory stimulation can be emitted by or transmitted from the mobile phone speakers, or any suitable accessory that can communicate with the mobile application such as earphones or headphones.

According to some embodiments, the auditory stimulation can be delivered at one or more frequencies, or within a range of frequencies. For example, the auditory stimulation can be set to be delivered at frequencies in any range, e.g., within an upper limit of about 20 kHz and a lower limit of about 20 Hz, inclusive. According to some embodiments, the stimulation can be set to be delivered at a frequency of about 20 Hz, about 30 Hz, about 40 Hz, about 50 Hz, about 60 Hz, about 70 Hz, about 80 Hz, about 90 Hz, about 100 Hz, about 125 Hz, about 150 Hz, about 200 Hz, about 300 Hz, about 400 Hz, about 500 Hz, about 600 Hz, about 700 Hz, about 800 Hz, about 900 Hz, about 1 kHz, about 1.5 kHz, about 2 kHz, about 2.5 kHz, about 3 kHz, about 3.5 kHz, about 4 kHz, about 4.5 kHz, about 5 kHz, about 5.5 kHz, about 6 kHz, about 6.5 kHz, about 7 kHz, about 7.5 kHz, about 8 kHz, about 8.5 kHz, about 9 kHz, about 9.5 kHz, about 10 kHz, about 11 kHz, about 12 kHz, about 13 kHz, about 14 kHz, about 15 kHz, about 16 kHz, about 17 kHz, about 18 kHz, about 19 kHz, or about 20 kHz, inclusive and any value in-between. According to some embodiments, the stimulation can be set to be delivered at frequencies from about 20 Hz to about 1 kHz, inclusive. According to some embodiments, the stimulation can be set to be delivered at frequencies from about 1 kHz to about 5 kHz, inclusive. Those of skill in the art will recognize that one or more of the above frequencies can be used as a boundary for a range of frequencies.

According to some embodiments, the auditory stimulation can be delivered at one or more decibels, or within a range of decibels. For example, the auditory stimulation can be set to be delivered at decibels in any range with an upper limit of about 200 dBA and a lower limit of about 0.1 dBA, inclusive. According to some embodiments, the auditory stimulation can be set to be delivered at a frequency of about 0.1 dBA, about 1 dBA, about 2 dBA, about 3 dBA, about 4 dBA, about 5 dBA, about 6 dBA, about 7 dBA, about 8 dBA, about 9 dBA, about 10 dBA, about 11 dBA, about 12 dBA, about 13 dBA, about 14 dBA, about 15 dBA, about 16 dBA, about 17 dBA, about 18 dBA, about 19 dBA, about 20 dBA, about 21 dBA, about 22 dBA, about 23 dBA, about 24 dBA, about 25 dBA, about 26 dBA, about 27 dBA, about 28 dBA, about 29 dBA, about 30 dBA, about 35 dBA, about 40 dBA, about 45 dBA, about 50 dBA, about 55 dBA, about 60 dBA, about 65 dBA, about 70 dBA, about 75 dBA, about 80 dBA, about 95 dBA, about 100 dBA, about 110 dBA, about 120 dBA, about 130 dBA, about 140 dBA, about 150 dBA, about 160 dBA, about 170 dBA, about 180 dBA, about 190 dBA, and about about 200 dBA, inclusive and any value in-between. According to some embodiments, the auditory stimulation can be delivered at frequencies from about 0.1 dBA to about 100 dBA, inclusive. According to some embodiments, the auditory stimulation can be set to be delivered at frequencies from about 0.1 dBA to about 85 dBA, inclusive. Those of skill in the art will recognize that one or more of the above frequencies can be used as a boundary for a range of frequencies.

According to some embodiments, somatic stimulation can include the emittance or transmittance of a mechanical or electrical haptic. A mechanical haptic may be transmitted to be felt in any form, including motion, vibration, buzz, pulse, beat, and the like and any combination thereof. An electrical haptic may be transmitted to be felt in any form, including an electrical conduction, and the like. Somatic stimulation can be emitted by or transmitted from the mobile phone, or any suitable accessory that can communicate with the mobile application, such as a pulse generator.

According to some embodiments, the somatic stimulation can be delivered at one or more vibrational frequencies, or within a range of frequencies. For example, the somatic stimulation can be set to be delivered at vibrational frequencies in any range, within an upper limit of about 300 Hz to about 0.1 Hz, inclusive. According to some embodiments, the stimulation can be set to be delivered at a frequency of about 0.1 Hz, about 10 Hz, about 20 Hz, about 30 Hz, about 40 Hz, about 50 Hz, about 55 Hz, about 60 Hz, about 65 Hz, about 70 Hz, about 75 Hz, about 80 Hz, about 95 Hz, about 100 Hz, about 105 Hz, about 110 Hz, about 115 Hz, about 120 Hz, about 125 Hz, about 130 Hz, about 135 Hz, about 140 Hz, about 145 Hz, about 150 Hz, about 155 Hz, about 160 Hz, about 165 Hz, about 170 Hz, about 175 Hz, about 180 Hz, about 185 Hz, about 190 Hz, about 195 Hz, about 200 Hz, about 210 Hz, about 220 Hz, about 230 Hz, about 240 Hz, about 250 Hz, about 260 Hz, about 270 Hz, about 280 Hz, about 290 Hz, about 300 Hz, inclusive and any value in between. According to some embodiments, the somatic stimulation can be set to be delivered at frequencies from about 0.1 Hz to about 200 Hz, inclusive. According to some embodiments, the somatic stimulation can be set to be delivered at frequencies from about 1 Hz to about 20 Hz, inclusive. Those of skill in the art will recognize that one or more of the above frequencies can be used as a boundary for a range of frequencies.

According to some embodiments, the somatic stimulation can be delivered at one or more electrical currents, or within a range of electrical currents. For example, the somatic stimulation can be set to be delivered at a current amount in any range within an upper limit of about 5 mA to about 0.1 μA, inclusive. According to some embodiments, the somatic stimulation can be set to be delivered at a current of about 0.1 μA, about 10 μA, about 20 μA, about 30 μA, about 40 μA, about 50 μA, about 60 μA, about 100 μA, about 200 μA, about 300 μA, about 400 μA, about 500 μA, about 600 μA, about 700 μA, about 800 μA, about 900 μA, about 1 mA, about 2 mA, about 3 mA, about 4 mA, or about 5 mA inclusive and any value in between. According to some embodiments, the somatic stimulation can be set to be delivered at currents from about 0.1 μA to about 100 μA, inclusive. According to some embodiments, the somatic stimulation can be set to be delivered at currents from about 1 μA to about 50 μA, inclusive. Those of skill in the art will recognize that one or more of the above currents can be used as a boundary of a range of currents.

According to some embodiments, the somatic stimulation can be delivered at one or more voltages, or within a range of voltages. For example, the somatic stimulation can be set to be delivered at a voltage in any range within an upper limit of about 50 V and a lower limit of 0.1 V, inclusive. According to some embodiments, the somatic stimulation can be set to be delivered at a voltage of about 0.1 V, about 1 V, about 2 V, about 3 V, about 4 V, about 5 V, about 6 V, about 7 V, about 8 V, about 9 V, about 10 V, about 11 V, about 12 V, about 13 V, about 4 V, about 15 V, about 16 V, about 7 V, about 18 V, about 19 V, about 20 V, about 21 V, about 22 V, about 23 V, about 24 V, about 25 V, about 26 V, about 27 V, about 28 V, about 29 V, about 30 V, about 31 V, about 32 V, about 33 V, about 34 V, about 35 V, about 36 V, about 37 V, about 38 V, about 39 V, about 40 V, about 41 V, about 42 V, about 43 V, about 44 V, about 45 V, about 46 V, about 47 V, about 48 V, about 49 V, or about 50 V. According to some embodiments, the somatic stimulation can be set to be delivered at a voltage from about 0.1 V to about 24 V, inclusive. According to some embodiments, the somatic stimulation can be set to be delivered at currents from about 1 V to about 5 V, inclusive. Those of skill in the art will recognize that one or more of the above voltages can be used as a boundary for a range of voltages.

According to some embodiments, olfactory stimulation can include the release of an odorant or the emittance or transmittance of any suitable stimulation that requires or elicits a subject to smell something, such as a candle, incense, essential oil, or object, or any combination of the above. For example, vestibular stimulation includes stimulation that is released and/or that elicits, instructs, or induces a subject to smell essential oils, such as lavender, lemon balm, peppermint, rosemary, phytoncide, valerian, jatamansi, holy basil, patchouli, geranium, chamomile, rose, vetivier, sweet basil, ylang ylang, frankincense, clary sage, marjoram, fennel, bergamot, and the like, or a smell that may be emotionally relevant to a subject, such as the smell of baked cookies, fresh bread, pine needles or a forest, freshly cut grass, raindrops on warm pavement, campfire smoke, a special perfume or cologne, and the like, or any combination of the above.

Olfactory stimulation, for example, can be induced by the release of olfactory stimulation or emission or by the transmission of visual or auditory stimulation to induce olfactory stimulation via the mobile device, or via any suitable accessory that communicate with the mobile application such as an essential oil diffuser.

According to some embodiments, olfactory stimulation can include the release of olfactory stimulation or emission or the transmission of visual or auditory stimulation to induce olfactory stimulation, or any combination of the above for at least about 5 seconds, at least about 10 seconds, at least about 20 seconds, at least about 30 seconds, at least about 1 minute, at least about 2 minute, at least about 3 minutes, at least about 4 minutes, at least about 5 minutes, at least about 6 minutes, at least about 7 minutes, at least about 8 minutes, at least about 9 minutes, at least about 10 minutes, at least about 20 minutes, at least about 30 minutes, at least about 40 minutes, at least about 50 minutes, at least about 1 hour, at least about 1.5 hours, or at least about 2 hours, inclusive and any value in between.

According to some embodiments, the olfactory stimulation can be set to be delivered for at least about 0.1 seconds to about 1 hour, inclusive. According to some embodiments, the olfactory stimulation can be set to be delivered for at least about 1 minute to at least about 30 minutes, inclusive.

Those of skill in the art will recognize that one or more of the above time ranges can be used as a boundary of time ranges.

According to some embodiments, the vestibular stimulation can include the emittance or transmittance of any suitable stimulation that requires or elicits a subject to physically move its head, portion of their body, its entire body, or any combination of the above. For example, vestibular stimulation includes stimulation that elicits, instructs, or induces a subject: to hum or chant, nod their head in an up-and-down manner; to shake its head in in an left-to-right manner; to move its upper body in an back-and-forth manner; to move its upper body in a side-to-side manner; to move its lower body in a back-and forth manner; to move its lower body in a side-to-side manner; move its feet or hands in a back and forth manner; move its feet or hands in a side-to-side manner; to cross its arms across their midline; clasp its hands, its opposing arms, its opposing shoulders, or opposite sides of the body across its midline; and any combination of the above.

Vestibular stimulation, for example, can be induced by the emission or the transmission of visual or auditory stimulation via the mobile device, or via any suitable accessory that can communicate with the mobile application such as headphones, earphones, and/or a microphone.

According to some embodiments, vestibular stimulation can include the emittance or transmittance of any suitable stimulation that requires a subject to physically move its head, portion of its body, its entire body, or any combination of the above for at least about 5 seconds, at least about 10 seconds, at least about 20 seconds, at least about 30 seconds, at least about 1 minute, at least about 2 minute, at least about 3 minutes, at least about 4 minutes, at least about 5 minutes, at least about 6 minutes, at least about 7 minutes, at least about 8 minutes, at least about 9 minutes, at least about 10 minutes, at least about 20 minutes, at least about 30 minutes, at least about 40 minutes, at least about 50 minutes, at least about 1 hour, at least about 1.5 hours, or at least about 2 hours, inclusive and any value in between.

According to some embodiments, the vestibular stimulation can be set to be delivered for at least about 0.1 seconds to about 1 hour, inclusive. According to some embodiments, the vestibular stimulation can be set to be delivered for at least about 1 minute to at least about 30 minutes, inclusive.

Those of skill in the art will recognize that one or more of the above time ranges can be used as a boundary of time ranges.

Delivery of Sensory Stimulation

According to some embodiments, the step of delivering at least one sensory stimulation comprises the delivery of the at least one sensory stimulation via the mobile device, any hardware component thereof, any software component thereof, and any external attachment or accessory thereof. A hardware component of the mobile device may be any hardware component suitable to deliver sensory stimulation, to interact with the user, and to communicate with the mobile device, any software component thereof, and/or any external accessories thereof, for example, a display, keyboard, haptics, speakers, microphone, and the like. A software component of the mobile device may be any software component suitable to deliver sensory stimulation, to interact with the user, and to communicate with the mobile device, any hardware component thereof, and/or any external accessories thereof, for example, wireless fidelity (“wi-fi”) capability, a data service, network connectivity, bluetooth capability, face recognition, thumbprint recognition, voice call, short message service (“SMS”), data call, data SMS, and the like. An external attachment or accessory of the mobile device may be any attachment or accessory suitable to deliver sensory stimulation, to interact with the user, and to communicate with the mobile device, any hardware components thereof, and/or any software component thereof, for example, speakers, headphones, microphone, and the like.

According to some embodiments, the step of delivering at least one sensory stimulation comprises the delivery (meaning sending forth, communicating, transmitting or emitting) of a therapeutic amount of sensory stimulation. According to some embodiments, a therapeutic amount of sensory stimulation may be calculated by the amount of sensory stimulation delivery required to induce a therapeutic effect, e.g., to evoke positive feelings or to reduce negative feelings of the user. According to some embodiments, a therapeutic amount of sensory stimulation may be specified as the length of the delivery of the at least one sensory stimulation, wherein the length is calculated by time. For example, the delivery of the at least one sensory stimulation may be for at least 0.1 second (“s”), 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 3 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute (“m”), 2 m, 3 m, 4 m, 5 m, 6 m, 7 m, 8 m, 9 m, 10 m, 11 m, 12 m, 13 m, 14 m, 15 m, 16 m, 17 m, 18 m, 19 m, 20 m, 21 m, 22 m, 23 m, 24 m, 25 m, 26 m, 27 m, 28 m, 29 m, 30 m, 31 m, 32 m, 33 m, 34 m, 35 m, 36 m, 37 m, 38 m, 39 m, 40 m, 41 m, 42 m, 43 m, 44 m, 45 m, 46 m, 47 m, 48 m, 49 m, 50 m, 51 m, 52 m, 53 m, 54 m, 55 m, 56 m, 57 m, 58 m, 59 m, 60 m, and the like, inclusive and any value in between.

According to some embodiments, the step of delivering at least one sensory stimulation further comprises the delivery of a therapeutic amount of the at least one sensory stimulation by any method that has a therapeutic effect on the user. According to some embodiments, the step of delivering at least one sensory stimulation comprises multiple methods of delivery that are effective to have a therapeutic effect on the user. According to some embodiments, the step of delivering at least one sensory stimulation comprises at least one method of delivery. According to some embodiments, the step of delivering at least one sensory stimulation further comprises the delivery of a pattern wherein the delivery of the pattern comprises repetition of at least one delivery method of the at least one sensory stimulation. According to some embodiments, the step of delivering at least one sensory stimulation comprises the delivery of a pattern wherein the delivery of the pattern comprises a therapeutic amount of repetitions of at least one delivery method of the at least one sensory stimulation. According to some embodiments, the step of delivering at least one sensory stimulation comprises the delivery of a plurality of patterns. According to some embodiments, the delivery of a pattern is rhythmic (meaning in a pattern of regular or irregular pulses by the occurrence of strong and weak melodic and harmonic beats), arrhythmic, or random. According to some embodiments, delivery of the pattern is an alternating pattern, i.e., delivery such that no two adjacent patterns are the same.

According to some embodiments, the step of delivering at least one sensory stimulation comprises the delivery of a therapeutic amount of a pattern. According to some embodiments, a therapeutic amount of a pattern may be calculated by the amount of sensory stimulation delivery required to induce a therapeutic effect on the user. According to some embodiments, a therapeutic amount of a pattern may be calculated by the amount of sensory stimulation required to induce a therapeutic effect, e.g., to evoke positive feelings or to reduce negative feelings of the user. According to some embodiments, a therapeutic amount of sensory stimulation may be specified as the length of the delivery of the pattern, wherein the length is calculated by time. For example, the delivery of the pattern may be for at least 0.1 second (“s”), 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 3 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute (“m”), 2 m, 3 m, 4 m, 5 m, 6 m, 7 m, 8 m, 9 m, 10 m, 11 m, 12 m, 13 m, 14 m, 15 m, 16 m, 17 m, 18 m, 19 m, 20 m, 21 m, 22 m, 23 m, 24 m, 25 m, 26 m, 27 m, 28 m, 29 m, 30 m, 31 m, 32 m, 33 m, 34 m, 35 m, 36 m, 37 m, 38 m, 39 m, 40 m, 41 m, 42 m, 43 m, 44 m, 45 m, 46 m, 47 m, 48 m, 49 m, 50 m, 51 m, 52 m, 53 m, 54 m, 55 m, 56 m, 57 m, 58 m, 59 m, 60 m, and the like, inclusive and any value in between.

According to some embodiments, the step of delivering at least one sensory stimulation comprises continuous (meaning uninterrupted in time) delivery, intermittent (meaning stopping or ceasing for a time and then beginning again) delivery, static (meaning fixed or stationary location) delivery, kinetic (meaning characterized by movement) delivery, unilateral (meaning occurring, relating to, or involving one side only) delivery, bilateral (meaning occurring, relating to or involving two sides) delivery, unilongitudinal (meaning superior or inferior) delivery, bilongitudinal (meaning superior and inferior) delivery, unicoronal (meaning anterior or posterior) delivery, and bicoronal (meaning anterior and posterior) delivery.

According to some embodiments, the step of delivering at least one sensory stimulation comprises continuous delivery of the at least one sensory stimulation. According to one aspect, the mobile application delivers continuously a visual stimulation, auditory stimulation, somatic stimulation, olfactory stimulation, and/or vestibular stimulation. For example, if the mobile application delivers visual stimulation continuously, according to some embodiments, the continuous visual delivery comprises one or more displayed lights or graphics that are continuously illuminated on the mobile device's screen or attachment thereof. In another example, if the mobile application delivers continuously an auditory stimulation, then the continuous auditory delivery comprises one or more emitted musical sounds or non-music sounds that are emanated continuously from the mobile device's speaker's or attachment thereof. In another example, if the mobile application delivers continuously a somatic stimulation, then the continuous somatic stimulation comprises one or more transmitted vibrations or electrical impulses that are transmitted continuously from the mobile device's screen or attachment thereof. In another example, if the mobile application delivers continuously an olfactory stimulation, then the continuous olfactory delivery comprises one or more sensory stimulation that is released, emitted or transmitted from the mobile device or attachment thereof to induce a user to smell an odorant. In another example, if the mobile application delivers continuously a vestibular stimulation, then the continuous vestibular delivery comprises one or more sensory stimulation that is emitted or transmitted from the mobile device or attachment thereof to induce a user to continuously physically move their body. According to some embodiments, the step of delivering at least one sensory stimulation comprises the delivery of a pattern comprising the repetitive continuous delivery of the at least one sensory stimulation. According to some embodiments, the step of delivering at least one sensory stimulation comprises the delivery of a pattern comprising the repetitive continuous delivery of the at least one sensory stimulation wherein a therapeutic amount of repetitions of continuous delivery is delivered.

According to some embodiments, the step of delivering at least one sensory stimulation comprises the intermittent delivery of the at least one sensory stimulation. According to one aspect, mobile application intermittently delivers visual stimulation, auditory stimulation, somatic stimulation, olfactory stimulation, and/or vestibular stimulation. For example, if the mobile application intermittently delivers visual stimulation, the intermittent visual delivery comprises one or more displayed lights or graphics that are periodically illuminated on the mobile device's screen or attachment thereof. In another example, if the mobile application intermittently delivers auditory stimulation, intermittent auditory delivery comprises one or more emitted musical sounds or non-music sounds that are periodically emanated from the mobile device's speakers or attachment thereof. In another example, if the mobile application intermittently delivers somatic stimulation, the intermittent somatic delivery comprises one or more transmitted vibrations or electrical impulses that are periodically transmitted from the mobile device's screen or attachment thereof. In another example, if the mobile application delivers intermittently an olfactory stimulation, then the intermittent olfactory delivery comprises one or more sensory stimulation that is released, emitted or transmitted from the mobile device or attachment thereof to induce a user to periodically smell an odorant. In another example, if the mobile application intermittently delivers vestibular stimulation, the intermittent vestibular delivery comprises one or more sensory stimulation that is emitted or transmitted from the mobile device or attachment thereof to induce a user to periodically physically move their body. According to some embodiments, the step of delivering at least one sensory stimulation comprises the delivery of a pattern comprising the repetitive intermittent delivery of the at least one sensory stimulation. According to some embodiments, the step of delivering at least one sensory stimulation comprises the delivery of a pattern comprising the repetitive intermittent delivery of the at least one sensory stimulation wherein a therapeutic amount of repetitions of intermittent delivery is delivered.

According to some embodiments, the step of delivering at least one sensory stimulation further comprises the static delivery of the at least one sensory stimulation. According to one aspect, mobile application statically delivers visual stimulation, auditory stimulation, somatic stimulation, olfactory stimulation, and/or vestibular stimulation. According to some embodiments, the static delivery of the at least one sensory stimulation induces the subject to perceive the stimulation to be originating from one spatial location. According to one aspect, the step of delivering at least one sensory stimulation comprises the static delivery of a visual stimulation, an auditory stimulation, a somatic stimulation, an olfactory stimulation, and/or vestibular stimulation. For example, if the step of delivering at least one sensory stimulation comprises the static delivery of visual stimulation, the static visual delivery comprises one or more displayed lights or graphics that are illuminated on the mobile device's screen or attachment thereof in a manner that induces the subject to perceive the stimulation to be originating from one spatial location. In another example, if the step of delivering at least one sensory stimulation comprises the static delivery of auditory stimulation, the static auditory delivery comprises one or more emitted musical sounds or non-musical sounds that are emitted from the mobile device's speakers or attachment thereof in a manner that induces the subject to perceive the stimulation to be originating from one spatial location. In another example, if the step of delivering at least one sensory stimulation comprises the static delivery of somatic stimulation, the static somatic delivery comprises one or more transmitted vibrations or electrical impulses that are transmitted from the mobile device's screen or attachment thereof in a manner that induces the subject to perceive the stimulation to be originating from one spatial location. In another example, if the step of delivering at least one sensory stimulation comprises the static delivery of olfactory stimulation, the static olfactory delivery comprises one or more sensory stimulation that is released, emitted or transmitted from the mobile device or attachment thereof in a manner that induces a user to smell an odorant in one spatial location or diffusing from one spatial location. In another example, if the step of delivering at least one sensory stimulation comprises the static delivery of vestibular stimulation, the static vestibular delivery comprises one or more sensory stimulation that is emitted or transmitted from the mobile device or attachment thereof in a manner that induces the subject to move their body but remain in one spatial location. According to some embodiments, the step of delivering at least one sensory stimulation comprises the delivery of a pattern comprising the repetitive static delivery of the at least one sensory stimulation. According to some embodiments, the step of delivering at least one sensory stimulation comprises the delivery of a pattern comprising the repetitive static delivery of the at least one sensory stimulation wherein a therapeutic amount of repetitions of static delivery is delivered.

According to some embodiments, the step of delivering at least one sensory stimulation comprises the kinetic delivery of the at least one sensory stimulation. According to one aspect, mobile application kinetically delivers visual stimulation, auditory stimulation, somatic stimulation, olfactory stimulation, and/or vestibular stimulation. According to some embodiments, the kinetic delivery of the at least one sensory stimulation induces the subject to perceive the stimulation to be originating from a first spatial location and then moving to a second spatial location. According to some embodiments, the movement of the sensory stimulation comprises linear movement or angular movement, wherein linear movement induces the subject to perceive all aspects of the stimulation to move in the same distance and direction at the same time, and wherein angular movement comprises induces the subject to perceive all aspects of the stimulation to move through the same angle and the same direction at the same time but not the same distance. According to some embodiments, linear movement may be rectilinear where the movement occurs in a straight line, or curvilinear, where the movement occurs in a curved path that isn't necessarily circular. According to some embodiments, linear movement may be horizontal wherein the movement occurs from the right direction to the left or from the left direction to the right. According to some embodiments, linear movement may be vertical wherein the movement occurs from the up direction to the down direction to the up direction. According to some embodiments, angular movement is also known as rotational movement. According to some embodiments, angular movement may be clockwise wherein the movement occurs towards the right direction. According to some embodiments, angular movement maybe counter-clockwise wherein the movement occurs towards the left direction. For example, if the step of delivering at least one sensory stimulation comprises the kinetic delivery of visual stimulation, the kinetic visual delivery comprises one or more displayed lights or graphics that are illuminated on the mobile device's screen or attachment thereof in a manner that induces the subject to perceive the stimulation to be originating from a first spatial location on the screen or attachment thereof and then linearly or angularly moving to a second spatial location on the screen or attachment thereof. In another example, if the step of delivering at least one sensory stimulation comprises the kinetic delivery of auditory stimulation, the kinetic auditory delivery comprises one or more emitted musical sounds or non-musical sounds that are emitted from the mobile device's speakers or attachment thereof in a manner that induces the subject to perceive the stimulation to be originating from a first spatial location from the speakers or attachment thereof and then linearly or angularly moving to a second spatial location from the speakers or attachment thereof. In another example, if the step of delivering at least one sensory stimulation comprises the kinetic delivery of somatic stimulation, the kinetic somatic delivery comprises one or more transmitted vibrations or electrical impulses that are transmitted from the mobile device's screen or attachment thereof in a manner that induces the subject to perceive the stimulation to be originating from a first spatial location on the screen or attachment thereof and then linearly or angularly moving to a second spatial location on the screen or attachment thereof. In another example, if the step of delivering at least one sensory stimulation comprises the kinetic delivery of olfactory stimulation, the kinetic olfactory delivery comprises one or more sensory stimulation that is released, emitted or transmitted from the mobile device or attachment thereof in a manner that induces a user to smell an odorant diffusing from a first spatial location from the mobile device or attachment thereof and then linearly or angularly diffusing to a second spatial location from the mobile device or attachment thereof. In another example, if the step of delivering at least one sensory stimulation comprises the kinetic delivery of vestibular stimulation, the kinetic vestibular delivery comprises one or more sensory stimulation that is emitted or transmitted from the mobile device or attachment thereof in a manner that induces the subject to linearly or angularly move their body from a first spatial location to a second spatial location. According to some embodiments, the step of delivering at least one sensory stimulation comprises the delivery of a pattern comprising the repetitive kinetic delivery of the at least one sensory stimulation. According to some embodiments, the step of delivering at least one sensory stimulation comprises the delivery of a pattern comprising the repetitive kinetic delivery of the at least one sensory stimulation wherein a therapeutic amount of repetitions of kinetic delivery is delivered. According to some embodiments, the step of delivering at least one sensory stimulation further comprises the unilateral delivery of the at least one sensory stimulation. According to some embodiments, the unilateral delivery of the at least one sensory stimulation induces the subject to perceive the stimulation via the left-hand side of the subject's respective sensory system or via the right-hand side of the subject's respective sensory system. According to one aspect, the step of delivering at least one sensory stimulation comprises the unilateral delivery of visual stimulation, auditory stimulation, somatic stimulation, olfactory stimulation, and/or vestibular stimulation. For example, if the step of delivering at least one sensory stimulation comprises the unilateral delivery of visual stimulation, the visual unilateral delivery comprises one or more displayed lights or graphics that are illuminated on the mobile device's screen or attachment thereof so that the subject is induced to perceive the stimulation via the left-hand side of the subject's visual sense system, such as the receptors in the subject's left eye, or via the right-hand side of the subject's visual sense system, such as the receptors in the subject's right eye. In another example, if the step of delivering at least one sensory stimulation comprises the unilateral delivery of auditory stimulation, the auditory unilateral delivery comprises one or more emitted musical sounds or non-music sounds that are emitted from the mobile device's speaker or attachment thereof so that the subject is induced to perceive the stimulation via the left-hand side of the subject's auditory sense system, such as the ear drum in the subject's left ear, or via the right-hand side of the subject's auditory sense system, such as the eardrum in the subject's right ear. In another example, if the step of delivering at least one sensory stimulation comprises the unilateral delivery of somatic stimulation, the somatic unilateral direction delivery comprises transmitting one or more vibrations or electrical impulses that are transmitted from the mobile device's screen or attachment thereof so that the subject is induced to perceive the stimulation via the left-hand side of the subject's somatic sense system, such as the fingertips on the subject's left hand, or via the right-hand side of the subject's somatic sense system, such as the fingertips on the subject's right hand. In another example, if the step of delivering at least one sensory stimulation comprises the unilateral delivery of olfactory stimulation, the unilateral olfactory delivery comprises one or more sensory stimulation that is released, emitted or transmitted from the mobile device or attachment thereof in a manner that so that the subject is induced to perceive the stimulation via the left-hand side of the subject's olfactory sense system, such as the subject's left-hand nostril, or via the right-hand side of the subject's olfactory sense system, such as the subject's right-hand nostril. In another example, if the step of delivering at least one sensory stimulation comprises the unilateral delivery of vestibular stimulation, the vestibular unilateral delivery comprises emitting or transmitting one or more sensory stimulations from the mobile device or attachment thereof so that the subject is induced to physically move the left-hand side of their body to stimulate their left-hand side of their vestibular sense system, such as the otolith organ in their left ear, or physically move the right-hand side of their body to stimulate the right-hand side of their vestibular sense system, such as the otolith organ in their right ear. According to some embodiments, the step of delivering at least one sensory stimulation comprises the delivery of a pattern comprising the repetitive unilateral delivery of the at least one sensory stimulation. According to some embodiments, the step of delivering at least one sensory stimulation comprises the delivery of a pattern comprising the repetitive unilateral delivery of the at least one sensory stimulation wherein a therapeutic amount of repetitions of unilateral delivery is delivered.

According to some embodiments, the step of delivering at least one sensory stimulation further comprises the bilateral delivery of the at least one sensory stimulation. According to some embodiments, the bilateral delivery of the at least one sensory stimulation induces the subject to perceive the stimulation via the left-hand side of the subject's respective sensory system and via the right-hand side of the subject's respective sensory system. According to some embodiments, the bilateral delivery of the at least one sensory stimulation simultaneously or alternatingly induces the subject to perceive the stimulation via their left-hand side sensory system and via their right-hand side sensory system. According to one aspect, the step of delivering at least one sensory stimulation comprises the bilateral delivery of visual stimulation, auditory stimulation, somatic stimulation, olfactory stimulation, and/or vestibular stimulation. For example, if the step of delivering at least one sensory stimulation comprises the bilateral delivery of visual stimulation, the visual bilateral delivery comprises one or more displayed lights or graphics that are illuminated on the mobile device's screen or attachment thereof so that the subject is induced to perceive the stimulation via the left-hand side of the subject's visual sense system, such as the receptors in the subject's left eye, and via the right-hand side of the subject's visual sense system, such as the receptors in the subject's right eye. In another example, if the step of delivering at least one sensory stimulation comprises the bilateral delivery of auditory stimulation, the auditory bilateral delivery comprises one or more emitted musical sounds or non-music sounds that are emitted from the mobile device's speaker or attachment thereof so that the subject is induced to perceive the stimulation via the left-hand side of the subject's auditory sense system, such as the ear drum in the subject's left ear, and via the right-hand side of the subject's auditory sense system, such as the eardrum in the subject's right ear. In another example, if the step of delivering at least one sensory stimulation comprises the bilateral delivery of somatic stimulation, the somatic bilateral direction delivery comprises transmitting one or more vibrations or electrical impulses that are transmitted from the mobile device's screen or attachment thereof so that the subject is induced to perceive the stimulation via the left-hand side of the subject's somatic sense system, such as the fingertips on the subject's left hand, and via the right-hand side of the subject's somatic sense system, such as the fingertips on the subject's right hand. In another example, if the step of delivering at least one sensory stimulation comprises the bilateral delivery of olfactory stimulation, the bilateral olfactory delivery comprises one or more sensory stimulation that is released, emitted or transmitted from the mobile device or attachment thereof in a manner that so that the subject is induced to perceive the stimulation via the left-hand side of the subject's olfactory sense system, such as the subject's left-hand nostril, and via the right-hand side of the subject's olfactory sense system, such as the subject's right-hand nostril. In another example, if the step of delivering at least one sensory stimulation comprises the bilateral delivery of vestibular stimulation, the vestibular bilateral delivery comprises emitting or transmitting one or more sensory stimulations from the mobile device or attachment thereof so that the subject is induced to physically move the left-hand side of their body to stimulate their left-hand side of their vestibular sense system, such as the otolith organ in their left ear, and physically move the right-hand side of their body to stimulate the right-hand side of their vestibular sense system, such as the otolith organ in their right ear. According to some embodiments, the step of delivering at least one sensory stimulation comprises the delivery of a pattern comprising the repetitive bilateral delivery of the at least one sensory stimulation. According to some embodiments, the step of delivering at least one sensory stimulation comprises the delivery of a pattern comprising the repetitive bilateral delivery of the at least one sensory stimulation wherein a therapeutic amount of repetitions of bilateral delivery is delivered.

According to some embodiments, a subject's senses can be stimulated via the sensory stimulation in a manner that can be alternated as stimulating from the subject's left side to right side. According to some embodiments, the frequency of the alternation from left to right, i.e., left-right-left-right etc., referred to herein as “laterality” can be tailored by the user. For example, laterality can switch every 0.1 second, 0.5 second, every 1 second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

According to some embodiments, the step of delivering at least one sensory stimulation further comprises the unilongitudinal delivery of the at least one sensory stimulation. According to some embodiments, the unilongitudinal delivery of the at least one sensory stimulation induces the subject to perceive the stimulation via the superior side of the subject's respective sensory system or via the inferior side of the subject's respective sensory system. According to one aspect, the step of delivering at least one sensory stimulation comprises the unilongitudinal delivery of visual stimulation, auditory stimulation, somatic stimulation, olfactory stimulation, and/or vestibular stimulation. For example, if the step of delivering at least one sensory stimulation comprises the unilongitudinal delivery of visual stimulation, the visual unilongitudinal delivery comprises one or more displayed lights or graphics that are illuminated on the mobile device's screen or attachment thereof so that the subject is induced to perceive the stimulation via the superior side of the subject's visual sense system, such as the receptors in the subject's eyes that perceive light in the superior direction of the visual field, or via the inferior side of the subject's visual sense system, such as the receptors in the subject's eyes that perceive light in the inferior direction of the visual field. In another example, if the step of delivering at least one sensory stimulation comprises the unilongitudinal delivery of auditory stimulation, the auditory unilongitudinal delivery comprises one or more emitted musical sounds or non-music sounds that are emitted from the mobile device's speaker or attachment thereof so that the subject is induced to perceive the stimulation via the superior side of the subject's auditory sense system, such emanating down the subject's ear canal to be perceived as coming from a superior direction, or via the inferior side of the subject's auditory sense system, such emanating up to the subject's ear canal to be perceived as coming from an inferior direction. In another example, if the step of delivering at least one sensory stimulation comprises the unilongitudinal delivery of somatic stimulation, the somatic unilongitudinal direction delivery comprises transmitting one or more vibrations or electrical impulses that are transmitted from the mobile device's screen or attachment thereof so that the subject is induced to perceive the stimulation via the superior side of the subject's somatic sense system, such transmitting vibrations that target the subject's face, or via the inferior side of the subject's somatic sense system, such transmitting vibrations that target the subject's stomach. In another example, if the step of delivering at least one sensory stimulation comprises the unilongitudinal delivery of olfactory stimulation, the unilongitudinal olfactory delivery comprises one or more sensory stimulation that is released, emitted or transmitted from the mobile device or attachment thereof in a manner that so that the subject is induced to perceive the stimulation via the superior side of the subject's olfactory sense system, such as diffusing from above the subject's head, or via the inferior side of the subject's olfactory sense system, such as diffusing from near the subject's feet. In another example, if the step of delivering at least one sensory stimulation comprises the unilongitudinal delivery of vestibular stimulation, the vestibular unilongitudinal delivery comprises emitting or transmitting one or more sensory stimulations from the mobile device or attachment thereof so that the subject is induced to physically move the superior side of their body to stimulate their superior side of their vestibular sense system, such as inducing the subject to nod their head to activate the otolith organs in their ears, or physically move the inferior side of their body to stimulate the inferior side of their vestibular sense system, such as inducing the subject to shuffle their feet to activate the sensory nerves located in the subject's ankles and feet. According to some embodiments, the step of delivering at least one sensory stimulation comprises the delivery of a pattern comprising the repetitive unilongitudinal delivery of the at least one sensory stimulation. According to some embodiments, the step of delivering at least one sensory stimulation comprises the delivery of a pattern comprising the repetitive unilongitudinal delivery of the at least one sensory stimulation wherein a therapeutic amount of repetitions of unilongitudinal delivery is delivered.

According to some embodiments, the step of delivering at least one sensory stimulation further comprises the bilongitudinal delivery of the at least one sensory stimulation. According to some embodiments, the bilongitudinal delivery of the at least one sensory stimulation induces the subject to perceive the stimulation via the superior side of the subject's respective sensory system and via the inferior side of the subject's respective sensory system. According to some embodiments, the bilongitudinal delivery of the at least one sensory stimulation simultaneously or alternatingly induces the subject to perceive the stimulation via their superior sensory system and via their inferior sensory system. According to one aspect, the step of delivering at least one sensory stimulation comprises the bilongitudinal delivery of visual stimulation, auditory stimulation, somatic stimulation, olfactory stimulation, and/or vestibular stimulation. For example, if the step of delivering at least one sensory stimulation comprises the bilongitudinal delivery of visual stimulation, the visual bilongitudinal delivery comprises one or more displayed lights or graphics that are illuminated on the mobile device's screen or attachment thereof so that the subject is induced to perceive the stimulation via the superior side of the subject's visual sense system, such as the receptors in the subject's eyes that perceive light in the superior direction of the visual field, and via the inferior side of the subject's visual sense system, such as the receptors in the subject's eyes that perceive light in the inferior direction of the visual field. In another example, if the step of delivering at least one sensory stimulation comprises the bilongitudinal delivery of auditory stimulation, the auditory bilongitudinal delivery comprises one or more emitted musical sounds or non-music sounds that are emitted from the mobile device's speaker or attachment thereof so that the subject is induced to perceive the stimulation via the superior side of the subject's auditory sense system, such emanating down the subject's ear canal to be perceived as coming from a superior direction, and via the inferior side of the subject's auditory sense system, such emanating up to the subject's ear canal to be perceived as coming from an inferior direction. In another example, if the step of delivering at least one sensory stimulation comprises the bilongitudinal delivery of somatic stimulation, the somatic bilongitudinal direction delivery comprises transmitting one or more vibrations or electrical impulses that are transmitted from the mobile device's screen or attachment thereof so that the subject is induced to perceive the stimulation via the superior side of the subject's somatic sense system, such as transmitting vibrations that target the subject's face, and via the inferior side of the subject's somatic sense system, such as transmitting vibrations that target the subject's abdomen. In another example, if the step of delivering at least one sensory stimulation comprises the bilongitudinal delivery of olfactory stimulation, the bilongitudinal olfactory delivery comprises one or more sensory stimulation that is released, emitted or transmitted from the mobile device or attachment thereof in a manner that so that the subject is induced to perceive the stimulation via the superior side of the subject's olfactory sense system, such as diffusing from above the subject's head, and via the inferior side of the subject's olfactory sense system, such as diffusing from near the subject's feet. In another example, if the step of delivering at least one sensory stimulation comprises the bilongitudinal delivery of vestibular stimulation, the vestibular bilongitudinal delivery comprises emitting or transmitting one or more sensory stimulations from the mobile device or attachment thereof so that the subject is induced to physically move the superior side of their body to stimulate their superior side of their vestibular sense system, such as inducing the subject to nod their head to activate the otolith organs in their ears, and physically move the inferior side of their body to stimulate the inferior side of their vestibular sense system, such as inducing the subject to shuffle their feet to activate the sensory nerves located in the subject's ankles and feet. According to some embodiments, the step of delivering at least one sensory stimulation comprises the delivery of a pattern comprising the repetitive bilongitudinal delivery of the at least one sensory stimulation. According to some embodiments, the step of delivering at least one sensory stimulation comprises the delivery of a pattern comprising the repetitive bilongitudinal delivery of the at least one sensory stimulation wherein a therapeutic amount of repetitions of bilongitudinal delivery is delivered.

According to some embodiments, a subject's senses can be stimulated via the sensory stimulation in a manner that can be alternated as stimulating from the subject's superior side to inferior side. According to some embodiments, the frequency of the alternation from superior to inferior, i.e., superior-inferior-superior-inferior etc., referred to herein as “longitudinality” can be tailored by the user. For example, longitudinality can switch every second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

According to some embodiments, the step of delivering at least one sensory stimulation further comprises the unicoronal delivery of the at least one sensory stimulation. According to some embodiments, the unicoronal delivery of the at least one sensory stimulation induces the subject to perceive the stimulation via the anterior side of the subject's respective sensory system or via the posterior side of the subject's respective sensory system. According to one aspect, the step of delivering at least one sensory stimulation comprises the unicoronal delivery of visual stimulation, auditory stimulation, somatic stimulation, olfactory stimulation, and/or vestibular stimulation. For example, if the step of delivering at least one sensory stimulation comprises the unicoronal delivery of visual stimulation, the visual unicoronal delivery comprises one or more displayed lights or graphics that are illuminated on the mobile device's screen or attachment thereof so that the subject is induced to perceive the stimulation via the anterior side of the subject's visual sense system, such as the receptors that perceive light coming from the front of the subject's visual field, or via the posterior side of the subject's visual sense system, such as the receptors that perceive light coming from the periphery of the subject's visual field. In another example, if the step of delivering at least one sensory stimulation comprises the unicoronal delivery of auditory stimulation, the auditory unicoronal delivery comprises one or more emitted musical sounds or non-music sounds that are emitted from the mobile device's speaker or attachment thereof so that the subject is induced to perceive the stimulation via the anterior side of the subject's auditory sense system, such as emanating towards the subject's ear canal from in front of the subject to be perceived as coming from the front of the subject, or via the posterior side of the subject's auditory sense system, such as emanating towards the subject's ear canal from behind the subject to be perceived as coming from behind the subject. In another example, if the step of delivering at least one sensory stimulation comprises the unicoronal delivery of somatic stimulation, the somatic unicoronal direction delivery comprises transmitting one or more vibrations or electrical impulses that are transmitted from the mobile device's screen or attachment thereof so that the subject is induced to perceive the stimulation via the anterior side of the subject's somatic sense system, such as transmitting vibrations that target the subject's abdomen, or via the posterior side of the subject's somatic sense system, such as transmitting vibrations that target the subject's back. In another example, if the step of delivering at least one sensory stimulation comprises the unicoronal delivery of olfactory stimulation, the unicoronal olfactory delivery comprises one or more sensory stimulation that is released, emitted or transmitted from the mobile device or attachment thereof in a manner that so that the subject is induced to perceive the stimulation via the anterior side of the subject's olfactory sense system, such as diffusing from in front of the subject, or via the posterior side of the subject's olfactory sense system, such as diffusing from behind the subject. In another example, if the step of delivering at least one sensory stimulation comprises the unicoronal delivery of vestibular stimulation, the vestibular unicoronal delivery comprises emitting or transmitting one or more sensory stimulations from the mobile device or attachment thereof so that the subject is induced to physically move the anterior side of their body to stimulate their anterior side of their vestibular sense system, such as receptors on their face, or physically move the posterior side of their body to stimulate the posterior side of their vestibular sense system, such as the receptors near their spine. According to some embodiments, the step of delivering at least one sensory stimulation comprises the delivery of a pattern comprising the repetitive unicoronal delivery of the at least one sensory stimulation. According to some embodiments, the step of delivering at least one sensory stimulation comprises the delivery of a pattern comprising the repetitive unicoronal delivery of the at least one sensory stimulation wherein a therapeutic amount of repetitions of unicoronal delivery is delivered.

According to some embodiments, the step of delivering at least one sensory stimulation further comprises the bicoronal delivery of the at least one sensory stimulation. According to some embodiments, the bicoronal delivery of the at least one sensory stimulation induces the subject to perceive the stimulation via the anterior side of the subject's respective sensory system and via the posterior side of the subject's respective sensory system. According to some embodiments, the bicoronal delivery of the at least one sensory stimulation simultaneously or alternatingly induces the subject to perceive the stimulation via their anterior sensory system and via their posterior sensory system. According to one aspect, the step of delivering at least one sensory stimulation comprises the bicoronal delivery of visual stimulation, auditory stimulation, somatic stimulation, olfactory stimulation, and/or vestibular stimulation. For example, if the step of delivering at least one sensory stimulation comprises the bicoronal delivery of visual stimulation, the visual bicoronal delivery comprises one or more displayed lights or graphics that are illuminated on the mobile device's screen or attachment thereof so that the subject is induced to perceive the stimulation via the anterior side of the subject's visual sense system, such as the receptors that perceive light coming from the front of the subject's visual field, and via the posterior side of the subject's visual sense system, such as the receptors that perceive light coming from the periphery of the subject's visual field. In another example, if the step of delivering at least one sensory stimulation comprises the bicoronal delivery of auditory stimulation, the auditory bicoronal delivery comprises one or more emitted musical sounds or non-music sounds that are emitted from the mobile device's speaker or attachment thereof so that the subject is induced to perceive the stimulation via the anterior side of the subject's auditory sense system, such as emanating towards the subject's ear canal from in front of the subject to be perceived as coming from the front of the subject, and via the posterior side of the subject's auditory sense system, such as emanating towards the subject's ear canal from behind the subject to be perceived as coming from behind the subject. In another example, if the step of delivering at least one sensory stimulation comprises the bicoronal delivery of somatic stimulation, the somatic bicoronal direction delivery comprises transmitting one or more vibrations or electrical impulses that are transmitted from the mobile device's screen or attachment thereof so that the subject is induced to perceive the stimulation via the anterior side of the subject's somatic sense system, such as transmitting vibrations that target the subject's abdomen, and via the posterior side of the subject's somatic sense system, such as transmitting vibrations that target the subject's back. In another example, if the step of delivering at least one sensory stimulation comprises the bicoronal delivery of olfactory stimulation, the bicoronal olfactory delivery comprises one or more sensory stimulation that is released, emitted or transmitted from the mobile device or attachment thereof in a manner that so that the subject is induced to perceive the stimulation via the anterior side of the subject's olfactory sense system, such as diffusing from in front of the subject, and via the posterior side of the subject's olfactory sense system, such as diffusing from behind the subject. In another example, if the step of delivering at least one sensory stimulation comprises the bicoronal delivery of vestibular stimulation, the vestibular bicoronal delivery comprises emitting or transmitting one or more sensory stimulations from the mobile device or attachment thereof so that the subject is induced to physically move the anterior side of their body to stimulate their anterior side of their vestibular sense system, such as receptors on their face, and physically move the posterior side of their body to stimulate the posterior side of their vestibular sense system, such as the receptors near their spine. According to some embodiments, the step of delivering at least one sensory stimulation comprises the delivery of a pattern comprising the repetitive bicoronal delivery of the at least one sensory stimulation. According to some embodiments, the step of delivering at least one sensory stimulation comprises the delivery of a pattern comprising the repetitive bicoronal delivery of the at least one sensory stimulation wherein a therapeutic amount of repetitions of bicoronal delivery is delivered.

According to some embodiments, a subject's senses can be stimulated via the sensory stimulation in a manner that can be alternated as stimulating from the subject's anterior side to posterior side. According to some embodiments, the frequency of the alternation from anterior to posterior, i.e., anterior-posterior-anterior-posterior etc., referred to herein as “coronality” can be tailored by the user. For example, coronality can switch every 0.1 second, 0.5 second, every 1 second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

According to some embodiments, the step of delivering at least one sensory stimulation further comprises repeated delivery of a pattern comprising repetition of at least one delivery method of at least one sensory stimulation. According to some embodiments, the repetition of at least one delivery method comprises a first commencement of a certain type of delivery (i.e., continuous, kinetic, etc.), cessation of that delivery after a therapeutic amount of time, and a second commencement of the same type of delivery. According to some embodiments, the repeated pattern is delivered for a therapeutic amount of time. According to some embodiments, the pattern may be repeated for an amount of time tailored by the user. According to some embodiments, the pattern is repeated every 0.1 second, 0.5 second, every 1 second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

For example, the repeated delivery of a pattern can comprise repetition of bilateral delivery of visual stimulation. According to some embodiments, repetition delivery of a pattern comprises a repetition of bilateral delivery of visual stimulation (i.e., alternately stimulating the left side and the right side) may be repeated every 0.1 second, 0.5 second, every 1 second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

According to some embodiments, more than one pattern may be different concurrently; for example, repeated delivery of a first pattern comprising repetition of bilateral delivery of visual stimulation with the concurrent delivery of a second pattern comprising repetition of continuous delivery of auditory stimulation.

According to some embodiments, the step of delivering at least one sensory stimulation further comprises an alternating delivery of a pattern comprising alternation of at least two delivery methods of at least one sensory stimulation. According to some embodiments, the alternation of at least one delivery method comprises a commencement of a first type of delivery (i.e., continuous, kinetic, etc.), the cessation of said first delivery after a therapeutic amount of time, a commencement of a second type of delivery different from the first type of delivery, and the cessation of said second delivery after a therapeutic amount of time. According to some embodiments, an alternating pattern is delivered for a therapeutic amount of time. According to some embodiments, the pattern may be alternated for an amount of time tailored by the user. According to some embodiments, the pattern is alternated every 0.1 second, 0.5 second, every 1 second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

For example, an alternating delivery of a pattern can comprise alternation of a left-hand side unilateral delivery of visual stimulation alternating with a right-hand side unilateral delivery (left-right-left-right-etc). According to some embodiments, an alternating delivery of a pattern can comprise a left-hand side unilateral delivery of visual stimulation alternating with and a right-hand side unilateral delivery (i.e., stimulating from left to right to left again) may be alternated every second, every 2 seconds, every 3 seconds, every 4 seconds, every 5 seconds, every 6 seconds, every 7 seconds, every 8 seconds, every 9 seconds, every 10 seconds, every 11 seconds, every 12 seconds, every 13 seconds, every 14 seconds, every 15 seconds, every 16 seconds, every 17 seconds, every 18 seconds, every 19 seconds, or every 20 seconds.

In another example, repeated delivery of a first pattern can comprise a left-hand side unilateral delivery of visual stimulation alternating with a right-hand side unilateral delivery concurrently with the continuous delivery of auditory stimulation.

According to some embodiments, the step of delivering at least one sensory stimulation comprises delivery of a cycle. According to some embodiments, the delivery of a cycle comprises commencement of the delivery of a pattern, the delivery of the pattern, and the cessation of the delivery of a pattern, wherein the complete delivery of the pattern defines the completion of one cycle of the pattern. According to some embodiments, the delivery of a cycle comprises a subject's perception of the pattern, wherein the subject's perception of the pattern defines the completion of one cycle of the pattern.

According to some embodiments, the step of delivering at least one sensory stimulation comprises delivery of a therapeutic amount of a cycle. According to some embodiments, a therapeutic amount of a cycle may be calculated by the amount of sensory stimulation delivery required to induce a therapeutic effect. According to some embodiments, a therapeutic amount of a cycle may be specified as the length of the delivery of the cycle, wherein the length is calculated by time. For example, the delivery of the cycle may be for at least 0.1 second (“s”), 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 3 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute (“m”), 2 m, 3 m, 4 m, 5 m, 6 m, 7 m, 8 m, 9 m, 10 m, 11 m, 12 m, 13 m, 14 m, 15 m, 16m, 17 m, 18 m, 19 m, 20 m, 21 m, 22 m, 23 m, 24 m, 25 m, 26 m, 27 m, 28 m, 29 m, 30 m, 31 m, 32 m, 33 m, 34 m, 35 m, 36 m, 37 m, 38 m, 39 m, 40 m, 41 m, 42 m, 43 m, 44 m, 45 m, 46 m, 47 m, 48 m, 49 m, 50 m, 51 m, 52 m, 53 m, 54 m, 55 m, 56 m, 57 m, 58 m, 59 m, 60 m, and the like, inclusive and any value in between.

According to some embodiments, where the step of delivering at least one sensory stimulation comprises a repeated bilateral delivery of sensory stimulation in a pattern, the first bilateral delivery of sensory stimulation comprises delivering sensory stimulation so that a subject is stimulated or perceives stimulation in their left-hand side, and their right-hand side senses back to their left-hand side senses again to restart the second bilateral delivery, so that the subject's perception of the pattern defines the completion of one cycle (i.e., left-right-left-being one cycle). For example, if the step of delivering of at least one sensory stimulation comprises a repeated bilateral delivery of visual stimulation in a pattern, the first bilateral delivery of visual stimulation comprises delivering visual stimulation so that a subject is stimulated or perceives stimulation in their left-hand side visual sense system to their right-hand side visual sense system back to their left-hand side visual senses again to restart the second bilateral delivery, and the subject's perception of the pattern defines the completion of one cycle.

According to some embodiments, the step of delivering at least one sensory stimulation comprises the delivery of more than one cycle. According to some embodiments, the step of delivering at least one sensory stimulation comprises the delivery of a therapeutic amount of cycles. For example, if the step of delivering at least one sensory stimulation comprises the delivery of a pattern wherein the delivery of a pattern comprises the repeated bilateral delivery of sensory stimulation, the completion of one cycle of the pattern is defined by the subject's perception of the visual stimulation from their left-hand sense system to their right-hand sense system and back to the left again. In another example, if the step of delivering at least one sensory stimulation comprises the delivery of a pattern, wherein the delivery of a pattern comprises the repeated bilongitudinal delivery of sensory stimulation, the completion of one cycle of the pattern is defined by the subject's perception of the visual stimulation from their superior sense system to their inferior sense system and back to the superior again. In another example, if the step of delivering at least one sensory stimulation comprises the delivery of a pattern, wherein the delivery of a pattern comprises the repeated bicoronal delivery of sensory stimulation, the completion of one cycle of the pattern is defined by the subject's perception of the visual stimulation from their anterior sense system to their posterior sense system and back to the anterior again.

According to some embodiments, the step of delivering at least one sensory stimulation comprises the delivery of a therapeutic amount of cycles, wherein the delivery of a therapeutic amount of cycles in a specific order defines the delivery of a sequence. According to some embodiments, the step of delivering at least one sensory stimulation further comprises delivering at least 1 sequence, wherein each sequence comprises 2 or more cycles delivered in a specific order. According to some embodiments, the step of delivering at least one sensory stimulation further comprising delivering at least 1 sequence, wherein each sequence comprises 2 or more cycles, 3 or more cycles, 4 or more cycles, 5 or more cycles, 6 or more cycles, 7 or more cycles, 8 or more cycles, 9 or more cycles, 10 or more cycles, 11 or more cycles, 12 or more cycles, 13 or more cycles, 14 or more cycles, 15 or more cycles, 16 or more cycles, 17 or more cycles, 18 or more cycles, 19 or more cycles, 20 or more cycles, 21 or more cycles, 22 or more cycles, 23 or more cycles, 24 or more cycles, 25 or more cycles, 26 or more cycles, 27 or more cycles, 28 or more cycles, 29 or more cycles, 30 or more cycles, 31 or more cycles, 32 or more cycles, 33 or more cycles, 34 or more cycles, 35 or more cycles, 36 or more cycles, 37 or more cycles, 38 or more cycles, 39 or more cycles, 40 or more cycles, 41 or more cycles, 42 or more cycles, 43 or more cycles, 44 or more cycles, 45 or more cycles, 46 or more cycles, 47 or more cycles, 48 or more cycles, 49 or more cycles, 50 or more cycles, 51 or more cycles, 52 or more cycles, 53 or more cycles, 54 or more cycles, 55 or more cycles, 56 or more cycles, 57 or more cycles, 58 or more cycles, 59 or more cycles, 60 or more cycles, 61 or more cycles, 62 or more cycles, 63 or more cycles, 64 or more cycles, 65 or more cycles, 66 or more cycles, 67 or more cycles, 68 or more cycles, 69 or more cycles, 70 or more cycles, 71 or more cycles, 72 or more cycles, 73 or more cycles, 74 or more cycles, 75 or more cycles, 76 or more cycles, 77 or more cycles, 78 or more cycles, 79 or more cycles, 80 or more cycles, 81 or more cycles, 82 or more cycles, 83 or more cycles, 84 or more cycles, 85 or more cycles, 86 or more cycles, 87 or more cycles, 88 or more cycles, 89 or more cycles, 90 or more cycles, 91 or more cycles, 92 or more cycles, 93 or more cycles, 94 or more cycles, 95 or more cycles, 96 or more cycles, 97 or more cycles, 98 or more cycles, 99 or more cycles, or 100 or more cycles, and the like, delivered in a specific order. For example, the step of delivering at least one sensory stimulation further comprising delivering 1 sequence, wherein the sequence comprises 3 cycles comprising a first cycle, a second cycle, and a third cycle, and the first cycle is delivered first, the second cycle is delivered second, and the third cycle is delivered third. According to some embodiments, the sequence comprises an alternating cycle, i.e., delivery such that no two adjacent cycles are the same.

According to some embodiments, the step of delivering at least one sensory stimulation comprises the delivery of a therapeutic amount of a sequence. According to some embodiments, a therapeutic amount of a sequence may be calculated by the amount of sensory stimulation delivery required to induce a therapeutic effect on the user. According to some embodiments, a therapeutic amount of a sequence may be calculated by the length of the delivery of the sequence, wherein the length is calculated by time. For example, the delivery of the sequence may be for at least 0.1 second (“s”), 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 3 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute (“m”), 2 m, 3 m, 4 m, 5 m, 6 m, 7 m, 8 m, 9 m, 10 m, 11 m, 12 m, 13 m, 14 m, 15 m, 16 m, 17 m, 18 m, 19 m, 20 m, 21 m, 22 m, 23 m, 24 m, 25 m, 26 m, 27 m, 28 m, 29 m, 30 m, 31 m, 32 m, 33 m, 34 m, 35 m, 36 m, 37 m, 38 m, 39 m, 40 m, 41 m, 42 m, 43 m, 44 m, 45 m, 46 m, 47 m, 48 m, 49 m, 50 m, 51 m, 52 m, 53 m, 54 m, 55 m, 56 m, 57 m, 58 m, 59 m, 60 m, and the like, inclusive and any value in between.

According to some embodiments, the step of delivering at least one sensory stimulation further comprising delivering at least 1 sequence, wherein each sequence comprises 2 or more cycles delivered in a specific order. According to some embodiments, the step of delivering at least one sensory stimulation further comprising delivering at least 1 sequence, at least 2 sequences, at least 3 sequences, at least 4 sequences, at least 5 sequences, at least 6 sequences, at least 7 sequences, at least 8 sequences, at least 9 sequences, at least 10 sequences, at least 11 sequences, at least 12 sequences, at least 13 sequences, at least 14 sequences, at least 15 sequences, at least 16 sequences, at least 17 sequences, at least 18 sequences, at least 19 sequences, at least 20 sequences, at least 21 sequences, at least 22 sequences, at least 23 sequences, at least 24 sequences, at least 25 sequences, at least 26 sequences, at least 27 sequences, at least 28 sequences, at least 29 sequences, at least 30 sequences, at least 31 sequences, at least 32 sequences, at least 33 sequences, at least 34 sequences, at least 35 sequences, at least 36 sequences, at least 37 sequences, at least 38 sequences, at least 39 sequences, at least 40 sequences, at least 41 sequences, at least 42 sequences, at least 43 sequences, at least 44 sequences, at least 45 sequences, at least 46 sequences, at least 47 sequences, at least 48 sequences, at least 49 sequences, at least 50 sequences, at least 51 sequences, at least 52 sequences, at least 53 sequences, at least 54 sequences, at least 55 sequences, at least 56 sequences, at least 57 sequences, at least 58 sequences, at least 59 sequences, at least 60 sequences, at least 61 sequences, at least 62 sequences, at least 63 sequences, at least 64 sequences, at least 65 sequences, at least 66 sequences, at least 67 sequences, at least 68 sequences, at least 69 sequences, at least 70 sequences, at least 71 sequences, at least 72 sequences, at least 73 sequences, at least 74 sequences, at least 75 sequences, at least 76 sequences, at least 77 sequences, at least 78 sequences, at least 79 sequences, at least 80 sequences, at least 81 sequences, at least 82 sequences, at least 863 sequences, at least 84 sequences, at least 85 sequences, at least 86 sequences, at least 87 sequences, at least 88 sequences, at least 89 sequences, at least 90 sequences, at least 91 sequences, at least 92 sequences, at least 93 sequences, at least 94 sequences, at least 95 sequences, at least 96 sequences, at least 97 sequences, at least 98 sequences, at least 99 sequences, at least or 100 sequences, at least and the like, wherein each sequence comprises 2 or more cycles delivered in a specific order. For example, the step of delivering at least one sensory stimulation further comprising delivering 2 sequences, comprising a first sequence and a second sequence, wherein the first sequence comprises 3 cycles comprising a first cycle, a second cycle, and a third cycle, and the first cycle is delivered first, the second cycle is delivered second, and the third cycle is delivered third, and wherein the second sequence comprises 4 cycles, comprising a first cycle, a second cycle, and a third cycle, and the first cycle is delivered first, the second cycle is delivered second, the third cycle is delivered third, and the fourth cycle is delivered fourth, and the first sequence is delivered first and the second sequence is delivered second.

### Interaction

According to some embodiments, the step of allowing a subject to interact with the mobile application comprises allowing at least one sensory stimulation to be delivered to the subject such that the subject is induced (meaning stimulated or caused) to see, hear, smell, feel or otherwise sense the at least one sensory stimulation being delivered. For example, if visual stimulation is delivered, the subject is induced to see the visual stimulation, if auditory stimulation is delivered, the subject is induced to hear the auditory stimulation, if olfactory stimulation is delivered, the subject is induced to smell the olfactory stimulation, if somatic stimulation is delivered, the subject is induced to feel the olfactory stimulation, if vestibular stimulation is delivered, the subject is induced to move to feel the vestibular stimulation.

According to some embodiments, the step of allowing a subject to interact with the mobile application further comprises allowing the subject, an agent of the subject, or both to tailor the mobile application.

According to some embodiments, the step of allowing a subject to interact with the mobile application further comprises allowing the subject, agent, or both to tailor any one of the type and/or amount of sensory stimulation delivered, the type and/or amount of method of delivery of the sensory stimulation delivered, the type, order and/or the amount of patterns delivered, the order and/or the amount cycles delivered, and/or the order and/or amount of sequences delivered, multiples of the foregoing, or any combination thereof based on their personal preference. According to some embodiments, the subject, agent or both may tailor the type of sensory stimulation, the amount of sensory stimulation, or both. According to some embodiments, the subject, agent, or both may tailor the type of method of delivery, the amount of method of delivery, or both. According to some embodiments, the subject, agent, or both may tailor the type of pattern, the order of pattern(s), the amount of pattern(s), or all three. According to some embodiments, the subject, agent, or both may tailor the order of cycle(s), the amount of cycle(s), or both.

For example, the subject may tailor the mobile application to: deliver one or more types of sensory stimulation via one or more types of delivery methods without delivering it in any pattern, cycle, or sequence; deliver one or more types of sensory stimulation via one or more types of methods, and deliver the one or more types of sensory stimulation in one or more types of patterns without delivering it as a cycle, or sequence; deliver one or more types of sensory stimulation via one or more types of delivery methods, deliver the one or more types of sensory stimulation in one or more types of patterns, and deliver the one or more types of patterns in any number of cycles, without delivering it in any sequences; or deliver one or more types of sensory stimulation via one or more types of delivery methods, deliver the one or more types of sensory stimulation in one or more types of patterns, and deliver the one or more types of patterns in any number of cycles, and deliver the cycles in any number of sequences.

According to some embodiments, the subject may tailor the mobile application to deliver visual stimulation, auditory stimulation, somatic stimulation, olfactory stimulation, and/or vestibular stimulation, or any combination thereof. For example, the subject may tailor the mobile application to: deliver visual stimulation without delivering auditory, somatic, olfactory, or vestibular stimulation; deliver somatic stimulation without delivering visual, auditory, olfactory, or vestibular stimulation; deliver vestibular stimulation without delivering visual, auditory, or somatic stimulation; deliver olfactory stimulation without delivering visual, auditory, somatic or vestibular stimulation; deliver visual stimulation and somatic stimulation without delivering auditory, olfactory, or vestibular stimulation; deliver visual stimulation and auditory stimulation without delivering somatic, olfactory, or vestibular stimulation; deliver visual and olfactory stimulation without delivering auditory, somatic, or vestibular stimulation; deliver visual stimulation and vestibular stimulation without delivering auditory or somatic stimulation; deliver auditory and olfactory stimulation without delivering visual, somatic or vestibular stimulation; deliver auditory stimulation and somatic stimulation without delivering visual, olfactory, or vestibular stimulation; deliver auditory stimulation and vestibular stimulation without delivering somatic, olfactory, or vestibular stimulation; deliver olfactory, and somatic stimulation without delivering visual, auditory or vestibular stimulation; deliver olfactory and vestibular stimulation without delivering visual, auditory or somatic stimulation; deliver vestibular stimulation and somatic stimulation without delivering visual, olfactory, or auditory stimulation; deliver visual stimulation, auditory stimulation, and somatic stimulation without delivering olfactory or vestibular stimulation; deliver visual stimulation, auditory stimulation, and olfactory stimulation, without delivering somatic or vestibular stimulation; deliver visual stimulation, auditory stimulation, and somatic stimulation without delivering olfactory or vestibular stimulation; deliver visual stimulation, auditory stimulation, and vestibular stimulation without delivering somatic or olfactory stimulation; deliver visual stimulation, somatic stimulation, and vestibular stimulation without delivering auditory or olfactory stimulation; deliver auditory stimulation, somatic stimulation, and vestibular stimulation without delivering visual or olfactory stimulation; deliver auditory stimulation, somatic stimulation, and vestibular stimulation without delivering visual or olfactory stimulation; deliver auditory stimulation, vestibular stimulation, and visual stimulation without delivering somatic or olfactory stimulation; deliver visual, auditory, and olfactory stimulation without delivering somatic or vestibular stimulation; deliver visual, somatic and olfactory stimulation without delivering auditory or vestibular stimulation; deliver visual, vestibular and olfactory stimulation without delivering auditory or somatic stimulation; deliver auditory, somatic and olfactory stimulation without delivering visual or vestibular stimulation; deliver auditory, vestibular and olfactory stimulation without delivering visual or somatic stimulation; deliver somatic, olfactory and vestibular stimulation without delivery visual or auditory stimulation; deliver visual, auditory, somatic and olfactory stimulation without delivering vestibular stimulation; deliver visual, auditory, somatic and vestibular stimulation without delivering olfactory stimulation; deliver visual, somatic, olfactory and vestibular stimulation without delivering auditory stimulation; deliver auditory, somatic, olfactory, and vestibular stimulation without delivering visual stimulation; or deliver visual, auditory, somatic, olfactory and vestibular stimulation.

According to some embodiments, the subject may tailor the mobile application to deliver more than one type of visual stimulation, auditory stimulation, somatic stimulation, and/or vestibular stimulation, or any combination thereof, wherein visual stimulation comprises lights and graphics, wherein auditory stimulation comprises musical sounds and non-musical sounds, wherein somatic stimulation comprises vibrations and electrical impulses, wherein olfactory comprises releasing odorants and eliciting odorant release, and wherein vestibular stimulations comprises eliciting peripheral or central movements. For example, the subject may tailor the mobile application to: deliver more than one type of visual stimulation comprising lights and graphics without delivering auditory, somatic, olfactory, or vestibular stimulation; deliver more than one type of somatic stimulation comprising vibrations and electrical impulses without delivering visual, auditory, olfactory, or vestibular stimulation; deliver more than one type of olfactory stimulation comprising releasing odorants and eliciting odorant diffusion without delivering visual, auditory, somatic, or vestibular stimulation; deliver more than one type of vestibular stimulation comprising eliciting peripheral and central movements without delivering visual, auditory, olfactory, or somatic stimulation; deliver more than one type of visual stimulation comprising lights and graphics and somatic stimulation comprising vibrations and electrical impulses without delivering auditory, olfactory or vestibular stimulation; deliver more than one type of visual stimulation comprising lights and graphics and auditory stimulation comprising musical sounds and non-musical sounds without delivering somatic, olfactory, or vestibular stimulation; deliver more than one type of visual stimulation comprising lights and graphics and olfactory stimulation comprising releasing odorants and eliciting odorant diffusion without delivering auditory, somatic, or vestibular stimulation; deliver more than one type of visual stimulation comprising lights and graphics and vestibular stimulation comprising eliciting peripheral and central movements without delivering auditory somatic, or olfactory stimulation; deliver more than one type of auditory stimulation comprising musical sounds and non-musical sounds and somatic stimulation comprising vibrations and electrical impulses without delivering visual, olfactory, or vestibular stimulation; deliver more than one type of auditory stimulation comprising musical sounds and non-musical sounds and olfactory stimulation comprising odorant release and eliciting odorant diffusion without delivering visual, somatic, or vestibular stimulation; deliver more than one type of auditory stimulation comprising musical sounds and non-musical sounds and vestibular stimulation comprising eliciting peripheral and central movements without delivering visual, somatic, or olfactory stimulation; deliver more than one type of somatic stimulation comprising vibrations and electrical impulses and olfactory stimulation comprising odorant release and eliciting odorant diffusion without delivering visual, auditory, or vestibular stimulation; deliver more than one type of somatic stimulation comprising vibrations and electrical impulses and vestibular stimulation comprising eliciting peripheral and central movements without delivering visual, auditory, and olfactory stimulation; deliver more than one type of olfactory stimulation comprising odorant release and eliciting odorant diffusion and vestibular stimulation comprising eliciting peripheral and central movements without delivering visual, auditory, or somatic stimulation; deliver more than one type of visual stimulation comprising lights and graphics, auditory stimulation comprising musical sounds and non-musical sounds, and somatic stimulation comprising vibrations and electrical impulses without delivering olfactory or vestibular stimulation; deliver more than one type of visual stimulation comprising lights and graphics, auditory stimulation comprising musical sounds and non-musical sounds, and olfactory stimulation comprising odorant release and eliciting odorant diffusion without delivering somatic or vestibular stimulation; deliver more than one type of visual stimulation comprising lights and graphics, auditory stimulation comprising musical sounds and non-musical sounds, and vestibular stimulation comprising eliciting peripheral and central movements without delivering somatic stimulation without delivering somatic or olfactory stimulation; deliver more than one type of visual stimulation comprising lights and graphics, somatic stimulation comprising vibrations and electrical impulses, and olfactory stimulation comprising odorant release and eliciting odorant diffusion without delivering auditory or vestibular stimulation; deliver more than one type of visual stimulation comprising lights and graphics, somatic stimulation comprising vibrations and electrical impulses, and vestibular stimulation comprising eliciting peripheral and central movements without delivering auditory or olfactory stimulation; deliver more than one type of visual stimulation comprising lights and graphics, olfactory stimulation comprising odorant release and eliciting odorant diffusion, and vestibular stimulation comprising eliciting peripheral and central movements without delivering auditory or somatic stimulation; deliver more than one type of auditory stimulation comprising musical sounds and non-musical sounds, somatic stimulation comprising vibrations and electrical impulses, and olfactory stimulation comprising odorant release and eliciting odorant diffusion without delivering visual or vestibular stimulation; deliver more than one type of auditory stimulation comprising musical sounds and non-musical sounds, somatic stimulation comprising vibrations and electrical impulses, and vestibular stimulation comprising eliciting peripheral or central movements without delivering visual or olfactory stimulation; deliver more than one type of auditory stimulation comprising musical sounds and non-musical sounds, olfactory stimulation comprising odorant release and eliciting odorant diffusion, and vestibular stimulation comprising eliciting peripheral and central movements without delivering visual or somatic stimulation; deliver more than one type of visual stimulation comprising lights and graphics, auditory stimulation comprising musical sounds and non-musical sounds, somatic stimulation comprising vibrations and electrical impulses, and olfactory stimulation comprising odorant release and eliciting odorant diffusion without delivering vestibular stimulation; deliver more than one type of visual stimulation comprising lights and graphics, auditory stimulation comprising musical sounds and non-musical sounds, somatic stimulation comprising vibrations and electrical impulses, and vestibular stimulation comprising eliciting peripheral and central movements without delivering olfactory stimulation; deliver more than one type of visual stimulation comprising lights and graphics, auditory stimulation comprising musical sounds and non-musical sounds, olfactory stimulation comprising odorant release and eliciting odorant diffusion, and vestibular stimulation comprising eliciting peripheral and central movements without delivering somatic stimulation; deliver more than one type of visual stimulation comprising lights and graphics, somatic stimulation comprising vibrations and electrical impulses, olfactory stimulation comprising odorant release and eliciting odorant diffusion, and vestibular stimulation comprising eliciting peripheral and central movements without delivering auditory stimulation; deliver more than one type of auditory stimulation comprising musical sounds and non-musical sounds, somatic stimulation comprising vibrations and electrical impulses, olfactory stimulation comprising odorant release and eliciting odorant diffusion, and vestibular stimulation comprising eliciting peripheral and central movements without delivering visual stimulation; or deliver more than one type of visual stimulation comprising lights and graphics, auditory stimulation comprising musical sounds and non-musical sounds, somatic stimulation comprising vibrations and electrical impulses, and olfactory stimulation comprising odorant release and eliciting odorant diffusion, and vestibular stimulation comprising eliciting peripheral and central movements.

According to some embodiments, the subject may also tailor the mobile application to deliver the sensory stimulation via continuous delivery, intermittent delivery, static delivery, kinetic delivery, unilateral delivery, bilateral delivery, unilongitudinal delivery, bilongitudinal delivery, unicoronal delivery, and/or bicoronal delivery, or any combination thereof. According to some embodiments, the subject may tailor the mobile application to deliver the sensory stimulation via: static delivery without kinetic delivery; kinetic delivery without static delivery; or kinetic delivery and static delivery. In another example, the subject may tailor the mobile application to deliver the sensory stimulation via: unilateral delivery, without bilateral delivery, unilongitudinal delivery, bilongitudinal delivery, unicoronal delivery, or bicoronal delivery; bilateral delivery, without unilateral delivery, unilongitudinal delivery, bilongitudinal delivery, unicoronal delivery, or bicoronal delivery; unilongitudinal delivery, without unilateral delivery, bilateral delivery, bilongitudinal delivery, unicoronal delivery, or bicoronal delivery; bilongitudinal delivery, without unilateral delivery, bilateral delivery, unilongitudinal delivery, unicoronal delivery, or bicoronal delivery; unicoronal delivery, without unilateral delivery, bilateral delivery, unilongitudinal delivery, bilongitudinal delivery, or bicoronal delivery; bicoronal delivery, without unilateral delivery, bilateral delivery, unilongitudinal delivery, bilongitudinal delivery, or unicoronal delivery; unilateral delivery and a bilateral delivery, without unilongitudinal delivery, bilongitudinal delivery, unicoronal delivery, or bicoronal delivery; unilateral delivery and a unilongitudinal delivery, without bilateral delivery, bilongitudinal delivery, unicoronal delivery, or bicoronal delivery; unilateral delivery and a bilongitudinal delivery, without bilateral delivery unilongitudinal delivery, unicoronal delivery, or bicoronal delivery; unilateral delivery and unicoronal delivery, without bilateral delivery, unilongitudinal delivery, bilongitudinal delivery, or bicoronal delivery; unilateral delivery and bicoronal delivery, without bilateral delivery, unilongitudinal delivery, bilongitudinal delivery, or unicoronal delivery; bilateral delivery and unicoronal delivery, without unilateral delivery, unilongitudinal delivery, bilongitudinal delivery, or bicoronal delivery; bilateral delivery and bicoronal delivery, without unilateral delivery, unilongitudinal delivery, bilongitudinal delivery, or unicoronal delivery; unilongitudinal delivery and unicoronal delivery, without unilateral delivery, bilateral delivery, bilongitudinal delivery, or bicoronal delivery; unilongitudinal delivery and unicoronal delivery, without unilateral delivery, bilateral delivery, bilongitudinal delivery, or unicoronal delivery; bilongitudinal delivery and unicoronal delivery, without unilateral delivery, bilateral delivery, unilongitudinal delivery, or bicoronal delivery; bilongitudinal delivery and bicoronal delivery, without unilateral delivery, bilateral delivery, unilongitudinal delivery, or unicoronal delivery; unicoronal delivery and bicoronal delivery, without unilateral delivery, bilateral delivery, unilongitudinal delivery, or bilongitudinal delivery; unilateral delivery, bilateral delivery, and unilongitudinal delivery, without bilongitudinal delivery, unicoronal delivery, or bicoronal delivery; unilateral delivery, bilateral delivery, and bilongitudinal delivery, without unilongitudinal delivery, unicoronal delivery, or bicoronal delivery; unilateral delivery, bilateral delivery, and unicoronal delivery without unilongitudinal delivery, bilongitudinal delivery, or bicoronal delivery; unilateral delivery, bilateral delivery, and bicoronal delivery without unilongitudinal delivery, bilongitudinal delivery, or unicoronal delivery; unilateral delivery, unilongitudinal delivery, and bilongitudinal delivery, without bilateral delivery, unicoronal delivery, or bicoronal delivery; unilateral delivery, unilongitudinal delivery, and unicoronal delivery without bilateral delivery, bilongitudinal delivery, or bicoronal delivery; unilateral delivery, unilongitudinal delivery, and bicoronal delivery without bilateral delivery, bilongitudinal delivery, or unicoronal delivery; unilateral delivery, bilongitudinal delivery, and unicoronal delivery without bilateral delivery, unilongitudinal delivery, or bicoronal delivery; unilateral delivery, bilongitudinal delivery, and bicoronal delivery without bilateral delivery, unilongitudinal delivery, or unicoronal delivery; unilateral delivery, unicoronal delivery, and bicoronal delivery without bilateral delivery, unilongitudinal delivery, or bilongitudinal delivery; bilateral delivery, unilongitudinal delivery, bilongitudinal delivery without unilateral delivery, unicoronal delivery, bicoronal delivery; bilateral delivery, unilongitudinal delivery, unicoronal delivery without unilateral delivery, bilongitudinal delivery, or bicoronal delivery; bilateral delivery, unilongitudinal delivery, bicoronal delivery without unilateral delivery, bilongitudinal delivery, or unicoronal delivery; bilateral delivery, bilongitudinal delivery, unicoronal delivery without unilateral delivery, unilongitudinal delivery, or bicoronal delivery; bilateral delivery, bilongitudinal delivery, bicoronal delivery without unilateral delivery, unilongitudinal delivery, unicoronal delivery; bilateral delivery, unicoronal delivery, and bicoronal delivery, without unilateral delivery, unilongitudinal delivery and bilongitudinal delivery; unilongitudinal delivery, bilongitudinal delivery, and unicoronal delivery, without unilateral delivery, bilateral delivery, or bicoronal delivery; unilongitudinal delivery, bilongitudinal delivery, and bicoronal delivery, without unilateral delivery, bilateral delivery, or unicoronal delivery; unilongitudinal delivery, unicoronal delivery, and bicoronal delivery, without unilateral delivery, bilateral delivery, and bilongitudinal delivery; bilongitudinal delivery, unicoronal delivery, and bicoronal delivery, without unilateral delivery, bilateral delivery and unilongitudinal delivery; unilateral delivery, bilateral delivery, unilongitudinal delivery, and bilongitudinal delivery, without unicoronal delivery, or bicoronal delivery; unilateral delivery, bilateral delivery, unilongitudinal delivery, and unicoronal delivery, without bilongitudinal delivery, or bicoronal delivery; unilateral delivery, bilateral delivery, unilongitudinal delivery, and bicoronal delivery, without bilongitudinal delivery, or unicoronal delivery; unilateral delivery, bilateral delivery, bilongitudinal delivery, unicoronal delivery, without unilongitudinal delivery or bicoronal delivery; unilateral delivery, bilateral delivery, bilongitudinal delivery, bicoronal delivery, without unilongitudinal delivery or unicoronal delivery; unilateral delivery, bilateral delivery, unicoronal delivery, bicoronal delivery, without unilongitudinal delivery or bilongitudinal delivery; unilateral delivery, unilongitudinal delivery, bilongitudinal delivery, unicoronal delivery, without bilateral delivery, bicoronal delivery; unilateral delivery, unilongitudinal delivery, bilongitudinal delivery, bicoronal delivery, without bilateral delivery or unilongitudinal delivery; unilateral delivery, unilongitudinal delivery, unicoronal delivery, bicoronal delivery, without bilateral delivery or unicoronal delivery; unilateral delivery, bilongitudinal delivery, unicoronal delivery, bicoronal delivery, without bilateral delivery or unilongitudinal delivery; bilateral delivery, unilongitudinal delivery, bilongitudinal delivery, and unicoronal delivery, without unilateral delivery or bicoronal delivery; bilateral delivery, unilongitudinal delivery, bilongitudinal delivery, and bicoronal delivery, without unilateral delivery or unicoronal delivery; bilateral delivery, unilongitudinal delivery, unicoronal delivery, bicoronal delivery, without unilateral delivery or bilongitudinal delivery; bilateral delivery, bilongitudinal delivery, unicoronal delivery, bicoronal delivery, without unilateral delivery or unilongitudinal delivery; unilongitudinal delivery, bilongitudinal delivery, unicoronal delivery, bicoronal delivery, without unilateral delivery or bilateral delivery; unilateral delivery, bilateral delivery, unilongitudinal delivery, bilongitudinal delivery, unicoronal delivery, without bicoronal delivery; unilateral delivery, bilateral delivery, unilongitudinal delivery, bilongitudinal delivery, bicoronal delivery, without unicoronal delivery; unilateral delivery, bilateral delivery, unilongitudinal delivery, unicoronal delivery, bicoronal delivery, without bilongitudinal delivery; unilateral delivery, bilateral delivery, bilongitudinal delivery, unicoronal delivery, bicoronal delivery, without unilongitudinal delivery; unilateral delivery, unilongitudinal delivery, bilongitudinal delivery, unicoronal delivery, bicoronal delivery, without bilateral delivery; bilateral delivery, unilongitudinal delivery, bilongitudinal delivery, unicoronal delivery, bicoronal delivery, without unilateral delivery; or unilateral delivery, bilateral delivery, unilongitudinal delivery, bilongitudinal delivery, unicoronal delivery, and bicoronal delivery.

According to some embodiments, the subject may tailor the mobile application to deliver visual stimulation, auditory stimulation, somatic stimulation, olfactory stimulation and/or vestibular stimulation, or any combination thereof, via continuous delivery, intermittent delivery, static delivery, kinetic delivery, unilateral delivery, bilateral delivery, unilongitudinal delivery, bilongitudinal delivery, unicoronal delivery, and/or bicoronal delivery, or any combination thereof. According to some embodiments, the subject may tailor the mobile application to: continuously, intermittently, statically, kinetically, unilaterally, bilaterally, unilongitudinally, bilongitudinally, unicoronally, or bicoronally deliver visual stimulation without delivering auditory, somatic, olfactory, or vestibular stimulation; continuously, intermittently, statically, kinetically, unilaterally, bilaterally, unilongitudinally, bilongitudinally, unicoronally, or bicoronally deliver somatic stimulation without delivering visual, auditory, olfactory, or vestibular stimulation; continuously, intermittently, statically, kinetically, unilaterally, bilaterally, unilongitudinally, bilongitudinally, unicoronally, or bicoronally deliver vestibular stimulation without delivering visual, auditory, or somatic stimulation; continuously, intermittently, statically, kinetically, unilaterally, bilaterally, unilongitudinally, bilongitudinally, unicoronally, or bicoronally deliver olfactory stimulation without delivering visual, auditory, somatic or vestibular stimulation; continuously, intermittently, statically, kinetically, unilaterally, bilaterally, unilongitudinally, bilongitudinally, unicoronally, or bicoronally deliver visual stimulation and somatic stimulation without delivering auditory, olfactory, or vestibular stimulation; continuously, intermittently, statically, kinetically, unilaterally, bilaterally, unilongitudinally, bilongitudinally, unicoronally, or bicoronally deliver visual stimulation and auditory stimulation without delivering somatic, olfactory, or vestibular stimulation; continuously, intermittently, statically, kinetically, unilaterally, bilaterally, unilongitudinally, bilongitudinally, unicoronally, or bicoronally deliver visual and olfactory stimulation without delivering auditory, somatic, or vestibular stimulation; continuously, intermittently, statically, kinetically, unilaterally, bilaterally, unilongitudinally, bilongitudinally, unicoronally, or bicoronally deliver visual stimulation and vestibular stimulation without delivering auditory or somatic stimulation; continuously, intermittently, statically, kinetically, unilaterally, bilaterally, unilongitudinally, bilongitudinally, unicoronally, or bicoronally deliver auditory and olfactory stimulation without delivering visual, somatic or vestibular stimulation; continuously, intermittently, statically, kinetically, unilaterally, bilaterally, unilongitudinally, bilongitudinally, unicoronally, or bicoronally deliver auditory stimulation and somatic stimulation without delivering visual, olfactory, or vestibular stimulation; continuously, intermittently, statically, kinetically, unilaterally, bilaterally, unilongitudinally, bilongitudinally, unicoronally, or bicoronally deliver auditory stimulation and vestibular stimulation without delivering somatic, olfactory, or vestibular stimulation; continuously, intermittently, statically, kinetically, unilaterally, bilaterally, unilongitudinally, bilongitudinally, unicoronally, or bicoronally deliver olfactory, and somatic stimulation without delivering visual, auditory or vestibular stimulation; continuously, intermittently, statically, kinetically, unilaterally, bilaterally, unilongitudinally, bilongitudinally, unicoronally, or bicoronally deliver olfactory and vestibular stimulation without delivering visual, auditory or somatic stimulation; continuously, intermittently, statically, kinetically, unilaterally, bilaterally, unilongitudinally, bilongitudinally, unicoronally, or bicoronally deliver vestibular stimulation and somatic stimulation without delivering visual, olfactory, or auditory stimulation; continuously, intermittently, statically, kinetically, unilaterally, bilaterally, unilongitudinally, bilongitudinally, unicoronally, or bicoronally deliver visual stimulation, auditory stimulation, and somatic stimulation without delivering olfactory or vestibular stimulation; continuously, intermittently, statically, kinetically, unilaterally, bilaterally, unilongitudinally, bilongitudinally, unicoronally, or bicoronally deliver visual stimulation, auditory stimulation, and olfactory stimulation, without delivering somatic or vestibular stimulation; continuously, intermittently, statically, kinetically, unilaterally, bilaterally, unilongitudinally, bilongitudinally, unicoronally, or bicoronally deliver visual stimulation, auditory stimulation, and somatic stimulation without delivering olfactory or vestibular stimulation; continuously, intermittently, statically, kinetically, unilaterally, bilaterally, unilongitudinally, bilongitudinally, unicoronally, or bicoronally deliver visual stimulation, auditory stimulation, and vestibular stimulation without delivering somatic or olfactory stimulation; continuously, intermittently, statically, kinetically, unilaterally, bilaterally, unilongitudinally, bilongitudinally, unicoronally, or bicoronally deliver visual stimulation, somatic stimulation, and vestibular stimulation without delivering auditory or olfactory stimulation; continuously, intermittently, statically, kinetically, unilaterally, bilaterally, unilongitudinally, bilongitudinally, unicoronally, or bicoronally deliver auditory stimulation, somatic stimulation, and vestibular stimulation without delivering visual or olfactory stimulation; continuously, intermittently, statically, kinetically, unilaterally, bilaterally, unilongitudinally, bilongitudinally, unicoronally, or bicoronally deliver auditory stimulation, somatic stimulation, and vestibular stimulation without delivering visual or olfactory stimulation; continuously, intermittently, statically, kinetically, unilaterally, bilaterally, unilongitudinally, bilongitudinally, unicoronally, or bicoronally deliver auditory stimulation, vestibular stimulation, and visual stimulation without delivering somatic or olfactory stimulation; continuously, intermittently, statically, kinetically, unilaterally, bilaterally, unilongitudinally, bilongitudinally, unicoronally, or bicoronally deliver visual, auditory, and olfactory stimulation without delivering somatic or vestibular stimulation; continuously, intermittently, statically, kinetically, unilaterally, bilaterally, unilongitudinally, bilongitudinally, unicoronally, or bicoronally deliver visual, somatic and olfactory stimulation without delivering auditory or vestibular stimulation; continuously, intermittently, statically, kinetically, unilaterally, bilaterally, unilongitudinally, bilongitudinally, unicoronally, or bicoronally deliver visual, vestibular and olfactory stimulation without delivering auditory or somatic stimulation; continuously, intermittently, statically, kinetically, unilaterally, bilaterally, unilongitudinally, bilongitudinally, unicoronally, or bicoronally deliver auditory, somatic and olfactory stimulation without delivering visual or vestibular stimulation; continuously, intermittently, statically, kinetically, unilaterally, bilaterally, unilongitudinally, bilongitudinally, unicoronally, or bicoronally deliver auditory, vestibular and olfactory stimulation without delivering visual or somatic stimulation; continuously, intermittently, statically, kinetically, unilaterally, bilaterally, unilongitudinally, bilongitudinally, unicoronally, or bicoronally deliver somatic, olfactory and vestibular stimulation without delivery visual or auditory stimulation; continuously, intermittently, statically, kinetically, unilaterally, bilaterally, unilongitudinally, bilongitudinally, unicoronally, or bicoronally deliver visual, auditory, somatic and olfactory stimulation without delivering vestibular stimulation; continuously, intermittently, statically, kinetically, unilaterally, bilaterally, unilongitudinally, bilongitudinally, unicoronally, or bicoronally deliver visual, auditory, somatic and vestibular stimulation without delivering olfactory stimulation; continuously, intermittently, statically, kinetically, unilaterally, bilaterally, unilongitudinally, bilongitudinally, unicoronally, or bicoronally deliver visual, somatic, olfactory and vestibular stimulation without delivering auditory stimulation; continuously, intermittently, statically, kinetically, unilaterally, bilaterally, unilongitudinally, bilongitudinally, unicoronally, or bicoronally deliver auditory, somatic, olfactory, and vestibular stimulation without delivering visual stimulation; or continuously, intermittently, statically, kinetically, unilaterally, bilaterally, unilongitudinally, bilongitudinally, unicoronally, or bicoronally deliver visual, auditory, somatic, olfactory and vestibular stimulation.

In another example, the subject may tailor the mobile application to: continuously, intermittently, statically, kinetically, unilaterally, bilaterally, unilongitudinally, bilongitudinally, unicoronally, or bicoronally deliver visual stimulation without delivering auditory, somatic, olfactory, or vestibular stimulation; continuously, intermittently, statically, kinetically, unilaterally, bilaterally, unilongitudinally, bilongitudinally, unicoronally, or bicoronally deliver somatic stimulation without delivering visual, auditory, olfactory, or vestibular stimulation; continuously, intermittently, statically, kinetically, unilaterally, bilaterally, unilongitudinally, bilongitudinally, unicoronally, or bicoronally deliver vestibular stimulation without delivering visual, auditory, or somatic stimulation; continuously, intermittently, statically, kinetically, unilaterally, bilaterally, unilongitudinally, bilongitudinally, unicoronally, or bicoronally deliver olfactory stimulation without delivering visual, auditory, somatic or vestibular stimulation; continuously, intermittently, statically, kinetically, unilaterally, bilaterally, unilongitudinally, bilongitudinally, unicoronally, or bicoronally deliver visual stimulation and continuously, intermittently, statically, kinetically, unilaterally, bilaterally, unilongitudinally, bilongitudinally, unicoronally, or bicoronally deliver somatic stimulation without delivering auditory, olfactory, or vestibular stimulation; continuously, intermittently, statically, kinetically, unilaterally, bilaterally, unilongitudinally, bilongitudinally, unicoronally, or bicoronally deliver visual stimulation, and continuously, intermittently, statically, kinetically, unilaterally, bilaterally, unilongitudinally, bilongitudinally, unicoronally, or bicoronally deliver auditory stimulation without delivering somatic, olfactory, or vestibular stimulation; continuously, intermittently, statically, kinetically, unilaterally, bilaterally, unilongitudinally, bilongitudinally, unicoronally, or bicoronally deliver visual and olfactory stimulation without delivering auditory, somatic, or vestibular stimulation; continuously, intermittently, statically, kinetically, unilaterally, bilaterally, unilongitudinally, bilongitudinally, unicoronally, or bicoronally deliver visual stimulation, and continuously, intermittently, statically, kinetically, unilaterally, bilaterally, unilongitudinally, bilongitudinally, unicoronally, or bicoronally deliver vestibular stimulation without delivering auditory or somatic stimulation; continuously, intermittently, statically, kinetically, unilaterally, bilaterally, unilongitudinally, bilongitudinally, unicoronally, or bicoronally deliver auditory and olfactory stimulation without delivering visual, somatic or vestibular stimulation; continuously, intermittently, statically, kinetically, unilaterally, bilaterally, unilongitudinally, bilongitudinally, unicoronally, or bicoronally deliver auditory stimulation, and continuously, intermittently, statically, kinetically, unilaterally, bilaterally, unilongitudinally, bilongitudinally, unicoronally, or bicoronally deliver somatic stimulation without delivering visual, olfactory, or vestibular stimulation; continuously, intermittently, statically, kinetically, unilaterally, bilaterally, unilongitudinally, bilongitudinally, unicoronally, or bicoronally deliver auditory stimulation, and continuously, intermittently, statically, kinetically, unilaterally, bilaterally, unilongitudinally, bilongitudinally, unicoronally, or bicoronally deliver vestibular stimulation without delivering somatic, olfactory, or vestibular stimulation; continuously, intermittently, statically, kinetically, unilaterally, bilaterally, unilongitudinally, bilongitudinally, unicoronally, or bicoronally deliver olfactory, and somatic stimulation without delivering visual, auditory or vestibular stimulation; continuously, intermittently, statically, kinetically, unilaterally, bilaterally, unilongitudinally, bilongitudinally, unicoronally, or bicoronally deliver olfactory and vestibular stimulation without delivering visual, auditory or somatic stimulation; continuously, intermittently, statically, kinetically, unilaterally, bilaterally, unilongitudinally, bilongitudinally, unicoronally, or bicoronally deliver vestibular stimulation, and continuously, intermittently, statically, kinetically, unilaterally, bilaterally, unilongitudinally, bilongitudinally, unicoronally, or bicoronally deliver somatic stimulation without delivering visual, olfactory, or auditory stimulation; continuously, intermittently, statically, kinetically, unilaterally, bilaterally, unilongitudinally, bilongitudinally, unicoronally, or bicoronally deliver visual stimulation, continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver somatic stimulation without delivering olfactory or vestibular stimulation; continuously, intermittently, statically, kinetically, unilaterally, bilaterally, unilongitudinally, bilongitudinally, unicoronally, or bicoronally deliver visual stimulation, continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver olfactory stimulation, without delivering somatic or vestibular stimulation; continuously, intermittently, statically, kinetically, unilaterally, bilaterally, unilongitudinally, bilongitudinally, unicoronally, or bicoronally deliver visual stimulation, continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver somatic stimulation without delivering olfactory or vestibular stimulation; continuously, intermittently, statically, kinetically, unilaterally, bilaterally, unilongitudinally, bilongitudinally, unicoronally, or bicoronally deliver visual stimulation, continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver vestibular stimulation without delivering somatic or olfactory stimulation; continuously, intermittently, statically, kinetically, unilaterally, bilaterally, unilongitudinally, bilongitudinally, unicoronally, or bicoronally deliver visual stimulation, continuously, intermittently, statically, kinetically, unilaterally, bilaterally, unilongitudinally, bilongitudinally, unicoronally, or bicoronally deliver somatic stimulation, continuously, intermittently, statically, kinetically, unilaterally, bilaterally, unilongitudinally, bilongitudinally, unicoronally, or bicoronally deliver vestibular stimulation without delivering auditory or olfactory stimulation; continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver somatic stimulation, continuously, intermittently, statically, kinetically, unilaterally, bilaterally, unilongitudinally, bilongitudinally, unicoronally, or bicoronally deliver vestibular stimulation without delivering visual or olfactory stimulation; continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver somatic stimulation, continuously, intermittently, statically, kinetically, unilaterally, bilaterally, unilongitudinally, bilongitudinally, unicoronally, or bicoronally deliver vestibular stimulation without delivering visual or olfactory stimulation; continuously, intermittently, statically, kinetically, unilaterally, bilaterally, unilongitudinally, bilongitudinally, unicoronally, or bicoronally deliver auditory stimulation, continuously, intermittently, statically, kinetically, unilaterally, bilaterally, unilongitudinally, bilongitudinally, unicoronally, or bicoronally deliver vestibular stimulation, continuously, intermittently, statically, kinetically, unilaterally, bilaterally, unilongitudinally, bilongitudinally, unicoronally, or bicoronally deliver visual stimulation without delivering somatic or olfactory stimulation; continuously, intermittently, statically, kinetically, unilaterally, bilaterally, unilongitudinally, bilongitudinally, unicoronally, or bicoronally deliver visual, auditory, and olfactory stimulation without delivering somatic or vestibular stimulation; continuously, intermittently, statically, kinetically, unilaterally, bilaterally, unilongitudinally, bilongitudinally, unicoronally, or bicoronally deliver visual, somatic and olfactory stimulation without delivering auditory or vestibular stimulation; continuously, intermittently, statically, kinetically, unilaterally, bilaterally, unilongitudinally, bilongitudinally, unicoronally, or bicoronally deliver visual, vestibular and olfactory stimulation without delivering auditory or somatic stimulation; continuously, intermittently, statically, kinetically, unilaterally, bilaterally, unilongitudinally, bilongitudinally, unicoronally, or bicoronally deliver auditory, somatic and olfactory stimulation without delivering visual or vestibular stimulation; continuously, intermittently, statically, kinetically, unilaterally, bilaterally, unilongitudinally, bilongitudinally, unicoronally, or bicoronally deliver auditory, vestibular and olfactory stimulation without delivering visual or somatic stimulation; continuously, intermittently, statically, kinetically, unilaterally, bilaterally, unilongitudinally, bilongitudinally, unicoronally, or bicoronally deliver somatic, olfactory and vestibular stimulation without delivering visual or auditory stimulation; continuously, intermittently, statically, kinetically, unilaterally, bilaterally, unilongitudinally, bilongitudinally, unicoronally, or bicoronally deliver visual, continuously, intermittently, statically, kinetically, unilaterally, bilaterally, unilongitudinally, bilongitudinally, unicoronally, or bicoronally deliver auditory, continuously, intermittently, statically, kinetically, unilaterally, bilaterally, unilongitudinally, bilongitudinally, unicoronally, or bicoronally deliver somatic and continuously, intermittently, statically, kinetically, unilaterally, bilaterally, unilongitudinally, bilongitudinally, unicoronally, or bicoronally deliver olfactory stimulation without delivering vestibular stimulation; continuously, intermittently, statically, kinetically, unilaterally, bilaterally, unilongitudinally, bilongitudinally, unicoronally, or bicoronally deliver visual, continuously, intermittently, statically, kinetically, unilaterally, bilaterally, unilongitudinally, bilongitudinally, unicoronally, or bicoronally deliver auditory, continuously, intermittently, statically, kinetically, unilaterally, bilaterally, unilongitudinally, bilongitudinally, unicoronally, or bicoronally deliver somatic and continuously, intermittently, statically, kinetically, unilaterally, bilaterally, unilongitudinally, bilongitudinally, unicoronally, or bicoronally deliver vestibular stimulation without delivering olfactory stimulation; continuously, intermittently, statically, kinetically, unilaterally, bilaterally, unilongitudinally, bilongitudinally, unicoronally, or bicoronally deliver visual, continuously, intermittently, statically, kinetically, unilaterally, bilaterally, unilongitudinally, bilongitudinally, unicoronally, or bicoronally deliver somatic, continuously, intermittently, statically, kinetically, unilaterally, bilaterally, unilongitudinally, bilongitudinally, unicoronally, or bicoronally deliver olfactory and continuously, intermittently, statically, kinetically, unilaterally, bilaterally, unilongitudinally, bilongitudinally, unicoronally, or bicoronally deliver vestibular stimulation without delivering auditory stimulation; continuously, intermittently, statically, kinetically, unilaterally, bilaterally, unilongitudinally, bilongitudinally, unicoronally, or bicoronally deliver auditory, continuously, intermittently, statically, kinetically, unilaterally, bilaterally, unilongitudinally, bilongitudinally, unicoronally, or bicoronally deliver somatic, continuously, intermittently, statically, kinetically, unilaterally, bilaterally, unilongitudinally, bilongitudinally, unicoronally, or bicoronally deliver olfactory, and continuously, intermittently, statically, kinetically, unilaterally, bilaterally, unilongitudinally, bilongitudinally, unicoronally, or bicoronally deliver vestibular stimulation without delivering visual stimulation; or continuously, intermittently, statically, kinetically, unilaterally, bilaterally, unilongitudinally, bilongitudinally, unicoronally, or bicoronally deliver visual, continuously, intermittently, statically, kinetically, unilaterally, bilaterally, unilongitudinally, bilongitudinally, unicoronally, or bicoronally deliver auditory, continuously, intermittently, statically, kinetically, unilaterally, bilaterally, unilongitudinally, bilongitudinally, unicoronally, or bicoronally deliver somatic, continuously, intermittently, statically, kinetically, unilaterally, bilaterally, unilongitudinally, bilongitudinally, unicoronally, or bicoronally deliver olfactory and continuously, intermittently, statically, kinetically, unilaterally, bilaterally, unilongitudinally, bilongitudinally, unicoronally, or bicoronally deliver vestibular stimulation. For example, the subject may tailor the mobile application to deliver intermittent visual stimulation and deliver continuous auditory stimulation. In another example, the subject may tailor the mobile application to deliver kinetic visual stimulation, and to deliver static auditory stimulation.

According to some embodiments, the subject may also tailor the mobile application to deliver one or more cycles. According to some embodiments, the one or more cycles may constitute a therapeutic amount of cycles. For example, the subject may tailor the mobile application to deliver 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, 21 cycles, 22 cycles, 23 cycles, 24 cycles, 25 cycles, 26 cycles, 27 cycles, 28 cycles, 29 cycles, 30 cycles, 31 cycles, 32 cycles, 33 cycles, 34 cycles, 35 cycles, 36 cycles, 37 cycles, 38 cycles, 39 cycles, 40 cycles, 41 cycles, 42 cycles, 43 cycles, 44 cycles, 45 cycles, 46 cycles, 47 cycles, 48 cycles, 49 cycles, 50 cycles, 51 cycles, 52 cycles, 53 cycles, 54 cycles, 55 cycles, 56 cycles, 57 cycles, 58 cycles, 59 cycles, 60 cycles, 61 cycles, 62 cycles, 63 cycles, 64 cycles, 65 cycles, 66 cycles, 67 cycles, 68 cycles, 69 cycles, 70 cycles, 71 cycles, 72 cycles, 3 cycles, 74 cycles, 75 cycles, 76 cycles, 77 cycles, 78 cycles, 79 cycles, 80 cycles, 81 cycles, 82 cycles, 863 cycles, 84 cycles, 85 cycles, 86 cycles, 87 cycles, 88 cycles, 89 cycles, 90 cycles, 91 cycles, 92 cycles, 93 cycles, 94 cycles, 95 cycles, 96 cycles, 97 cycles, 98 cycles, 99 cycles, or 100 cycles, inclusive, and the like.

According to some embodiments, the subject may also tailor the order in which the mobile application delivers the cycles. For example, if the subject tailors the mobile application to deliver a sequence comprising 3 cycles delivered in a specific order, wherein the cycles comprise a first cycle, a second cycle, and a third cycle, the subject may tailor the mobile application to deliver the first cycle first, the second cycle second, and the third cycle third, or otherwise.

According to some embodiments, the subject may also tailor the mobile application to deliver one or more sequences. According to some embodiments, the one or more sequences may constitute a therapeutic amount of sequences. For example, the subject may tailor the mobile application to deliver at least 1 sequence, at least 2 sequences, at least 3 sequences, at least 4 sequences, at least 5 sequences, at least 6 sequences, at least 7 sequences, at least 8 sequences, at least 9 sequences, at least 10 sequences, at least 11 sequences, at least 12 sequences, at least 13 sequences, at least 14 sequences, at least 15 sequences, at least 16 sequences, at least 17 sequences, at least 18 sequences, at least 19 sequences, at least 20 sequences, at least 21 sequences, at least 22 sequences, at least 23 sequences, at least 24 sequences, at least 25 sequences, at least 26 sequences, at least 27 sequences, at least 28 sequences, at least 29 sequences, at least 30 sequences, at least 31 sequences, at least 32 sequences, at least 33 sequences, at least 34 sequences, at least 35 sequences, at least 36 sequences, at least 37 sequences, at least 38 sequences, at least 39 sequences, at least 40 sequences, at least 41 sequences, at least 42 sequences, at least 43 sequences, at least 44 sequences, at least 45 sequences, at least 46 sequences, at least 47 sequences, at least 48 sequences, at least 49 sequences, at least 50 sequences, at least 51 sequences, at least 52 sequences, at least 53 sequences, at least 54 sequences, at least 55 sequences, at least 56 sequences, at least 57 sequences, at least 58 sequences, at least 59 sequences, at least 60 sequences, at least 61 sequences, at least 62 sequences, at least 63 sequences, at least 64 sequences, at least 65 sequences, at least 66 sequences, at least 67 sequences, at least 68 sequences, at least 69 sequences, at least 70 sequences, at least 71 sequences, at least 72 sequences, at least 73 sequences, at least 74 sequences, at least 75 sequences, at least 76 sequences, at least 77 sequences, at least 78 sequences, at least 79 sequences, at least 80 sequences, at least 81 sequences, at least 82 sequences, at least 863 sequences, at least 84 sequences, at least 85 sequences, at least 86 sequences, at least 87 sequences, at least 88 sequences, at least 89 sequences, at least 90 sequences, at least 91 sequences, at least 92 sequences, at least 93 sequences, at least 94 sequences, at least 95 sequences, at least 96 sequences, at least 97 sequences, at least 98 sequences, at least 99 sequences, at least or 100 sequences, inclusive and the like.

According to some embodiments, the subject may also tailor the order in which the mobile application delivers the sequences. For example, if the subject tailors the mobile application to deliver 2 sequences, the sequences comprise a first sequence and a second sequence, wherein the first sequence comprises 3 cycles comprising a first cycle, a second cycle, and a third cycle, and the first cycle is delivered first, the second cycle is delivered second, and the third cycle is delivered third, and wherein the second sequence comprises 4 cycles, comprising a first cycle, a second cycle, and a third cycle, and the first cycle is delivered first, the second cycle is delivered second, the third cycle is delivered third, and the fourth cycle is delivered fourth, the subject may tailor the mobile application to deliver the first sequence is first and the second sequence second, or otherwise.

According to some embodiments, the methods of the present invention provides a mobile application that interacts with a subject in various ways, for example, passively, actively, and physiologically, further comprises allowing a subject to choose their desired type of interaction. According to some embodiments, the step of allowing a subject to interact with the mobile application of the present invention comprises a subject passively interfacing with the mobile application. According to some embodiments, the step of allowing a subject to interact with the mobile application of the present invention comprises a subject actively interfacing with the mobile application. According to some embodiments, the step of allowing a subject to interact with the mobile application of the present invention comprises a subject physiologically interfacing with the mobile application. According to some embodiments, the step of allowing a subject to interact with the mobile application comprises requiring a subject to choose their level of interaction.

According to some embodiments, a subject passively interfaces with the mobile application by selecting a pre-programmed level of passive interaction, and allowing the mobile application to deliver one or more sensory stimulation without further interaction from the subject. In some embodiments, the passive interfacing is by facial recognition software (e.g., FaceID).

According to some embodiments, a subject actively interfaces with the mobile application by selecting a pre-programmed level of active interaction, and allowing the mobile application to deliver at least one sensory stimulation that requires further interaction in the form of active responses from the subject. According to some embodiments, each delivery of sensory stimulation can be varied based on the user's active response or preferences choice. For example, the duration, interval, intensity, frequency, color, pattern, and images displayed during the visual sequence presentation can be varied based on the user's response or preferences. In another example, the duration, interval, volume, type of sound emitted during the auditory sequence presentation can be varied based on the user's response or preferences. According to one aspect, the method further comprises the step of the mobile application tailoring sensory stimulation(s), method (s) of delivery, pattern(s), cycle(s), and sequence(s) according to the subject's active responses. According to some embodiments, the active responses can be measured through the mobile phone screen. According to some embodiments, the active responses can be measured through a mobile phone accessory configured to measure user responses and communicate with the mobile application. Examples of mobile phone accessories include a keyboard, an electronic pencil, a microphone, headphones and/or earphones, and the like.

According to some embodiments, a subject physiologically interfaces with the mobile application by selecting a pre-programmed level of physiological interaction, and allowing the mobile application to deliver at least one sensory stimulation based on physiological responses displayed by the subject during delivery. According to one aspect, the method further comprises the step of the mobile application tailoring sensory stimulation(s), method(s) of delivery, pattern(s), cycle(s), and sequence(s) according to the subject's physiological responses. According to some embodiments, the physiological responses can be measured through the mobile phone screen. According to some embodiments, the physiological responses can be measured through an attachment configured to measure physiological responses and communicate with the mobile application. According to some embodiments, the attachment is a device that measures galvanic skin responses, otherwise known as an electrodermal response, blood pressure, heart rate, respiration rate and intensity, skin conductivity, or other physiological parameters. According to one aspect, the method further comprises the step of the mobile application tailoring sequence delivery depending on the subject's physiological responses. Examples of attachments include a heart rate monitor, a blood pressure monitor, a respiration sensor or a pulse oximeter, an electrodermal activity device, and the like.

According to some embodiments, the method of the described invention can be combined with performing various relaxation techniques. According to some embodiments, the method of the described invention can further include the step of providing instruction for the user to perform various relaxation techniques. Exemplary relaxation techniques include, but are not limited to, meditation, exercise, aromatherapy, breathing techniques, and the like.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention.

### Example 1

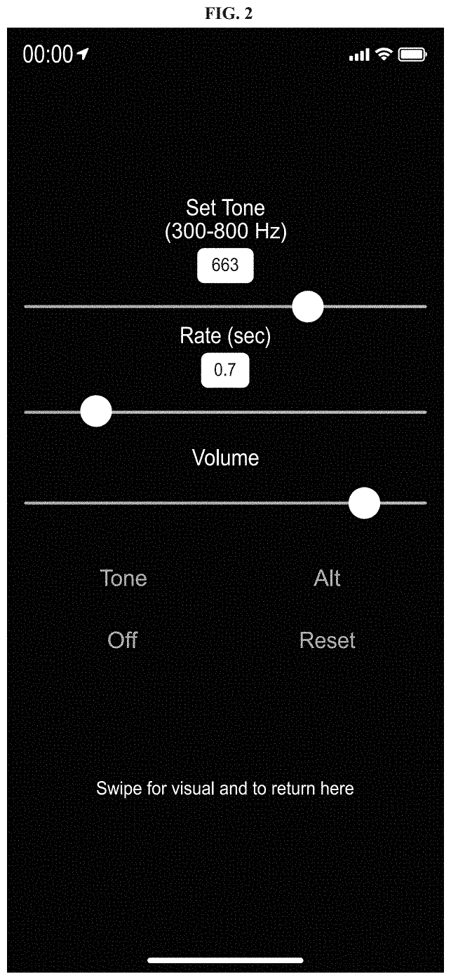

A mobile phone owner, such as the owner of an Apple® iPhone®, may download the mobile phone application described herein from the corresponding application store, such as the App Store for iPhone users. Once downloaded, the app user will open the app to access its features and run the program. The app user will interact with the phone by selecting auditory stimulation delivery and by tailoring the app to deliver the desired type of pattern(s), cycle(s), or sequence(s), the amount or length of the selected option(s), and/or the order of the selected option(s); alternatively the app user may select a preprogrammed option that delivers the auditory stimulation as previously determined by either the user or the mobile application itself. The user will turn on and adjust the volume of the mobile device as preferred and may play the auditory stimulation via the speakers in the mobile device or via headphones as connected to the mobile device either via cable or wirelessly such as by Bluetooth communication. The user will begin the selected program and the mobile application will deliver the auditory stimulation. The user has the option to tailor the sensory stimulation delivery throughout the delivery of the stimulation (FIG. 2).

### Example 2

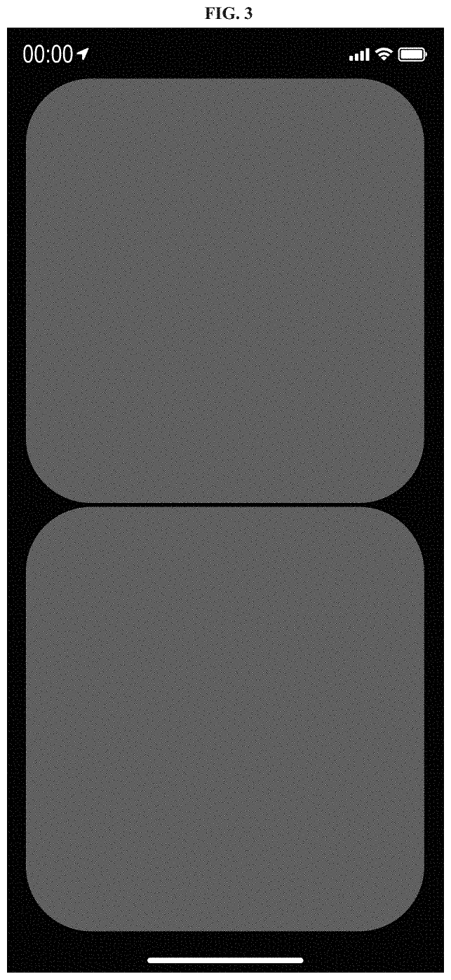

A mobile phone owner, such as the owner of an Apple® iPhone®, may download the mobile phone application described herein from the corresponding application store, such as the App Store for iPhone users. Once downloaded the app user will open the app to access its features and run the program. The appuser will interact with the phone by selecting auditory and visual stimulation delivery and by tailoring the app to deliver the desired type of pattern(s), cycle(s), or sequence(s), the amount or length of the selected option(s), and/or the order of the selected option(s); alternatively the app user may select a preprogrammed option that delivers the auditory and visual stimulation as previously determined by either the user or the mobile application itself. The user will turn on and adjust the volume of the mobile device as preferred and may play the auditory stimulation via the speakers in the mobile device or via headphones as connected to the mobile device either via cable or wirelessly such as by Bluetooth communication. The user will begin the selected program and the mobile application will deliver the auditory stimulation. The user has the option of tailoring the delivery throughout the delivery of the auditory stimulation (FIG. 2). The user may interact with the screen of the mobile device to access the delivery of the visual stimulation (FIG. 3).

### Example 3

A mobile phone owner, such as the owner of an Apple® iPhone®, may download the mobile phone application described herein from the corresponding application store, such as the App Store for iPhone users. Once downloaded the app user will open the app to access its features and run the program. The app user will interact with the phone by selecting visual stimulation delivery in a bilateral or bilongitudinal pattern and by tailoring the app to deliver the desired type of cycle(s), or sequence(s), the amount or length of the selected option(s), and/or the order of the selected option(s); alternatively the app user may select a preprogrammed option that delivers the visual stimulation delivery in a bilateral or bilongitudinal pattern as previously determined by either the user or the mobile application itself. The user may interact with the screen of the mobile device to access the delivery of the visual stimulation (FIG. 3) and can tailor the delivery of the visual pattern to be bilateral or bilongitudinal depending on the direction in which the user holds the phone, i.e., bilateral meaning that the user will hold the phone so that it is longer horizontally, and bilongitudinal meaning that the user will hold the phone so that it is longer vertically.

### Example 4

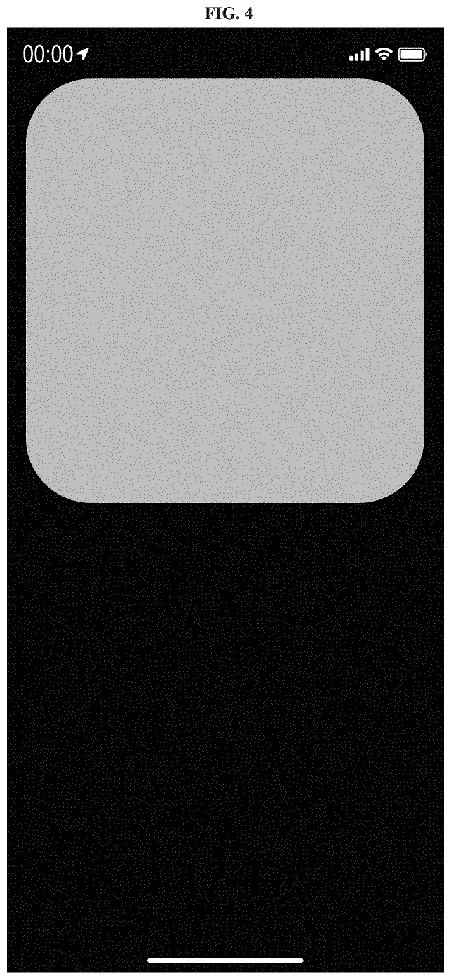

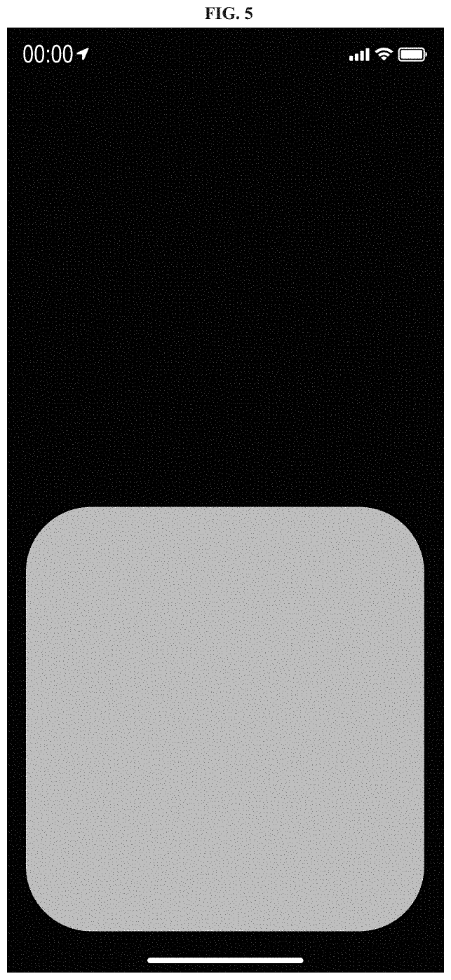

A mobile phone owner, such as the owner of an Apple® iPhone®, may download the mobile phone application described herein from the corresponding application store, such as the App Store for iPhone users. Once downloaded the app user will open the app to access its features and run the program. The app user will interact with the phone by selecting visual stimulation delivery in an intermittent bilateral or bilongitudinal pattern and by tailoring the app to deliver the desired type of cycle(s), or sequence(s), the amount or length of the selected option(s), and/or the order of the selected option(s); alternatively the app user may select a preprogrammed option that delivers the visual stimulation delivery in an intermittent bilateral or bilongitudinal pattern as previously determined by either the user or the mobile application itself. The user may interact with the screen of the mobile device to access the delivery of the visual stimulation in an intermittent bilateral or bilongitudinal pattern (FIGS. 4 and 5) and will also tailor the delivery of the pattern to be bilateral or bilongitudinal depending on the direction in which the user holds the phone, i.e., bilateral meaning that the user will hold the phone so that it is longer horizontally, and bilongitudinal meaning that the user will hold the phone so that it is longer vertically.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. A method for improving a perception of wellness by a subject comprising:
(a) providing a wellness improving mobile application, wherein the mobile application is configured to communicate with a mobile device or attachment thereof and is designed to deliver at least one sensory stimulation to a subject,
(b) delivering the at least one sensory stimulation to the subject, and
(c) the subject interacting with the mobile application, and
(d) the interacting in (c) improving the perception of wellness by the subject;
wherein the at least one sensory stimulation comprises visual stimulation, auditory stimulation, somatic stimulation, olfactory stimulation, or vestibular stimulation.

2. The method according to claim 1, wherein the mobile application delivering the sensory stimulation via continuous delivery, intermittent delivery, static delivery, kinetic delivery, unilateral delivery, bilateral delivery, unilongitudinal delivery, bilongitudinal delivery, unicoronal delivery, and bicoronal delivery or any combination thereof.

3. The method according to claim 1, wherein the delivering of the sensory stimulation is in a pattern, a cycle, a sequence or any combination thereof.

4. The method according to claim 1, wherein the interacting in (c) comprises inducing the subject to sense the sensory stimulation.

5. The method according to claim 1, wherein the interacting in (c) comprises actively, passively, or physiologically tailoring the interaction by tailoring: the type or amount of sensory stimulation delivered or both, the type or amount of method of delivery of the sensory stimulation delivered or both, the type, the order or the amount of patterns delivered or all three, the order or the amount cycles delivered or both, and the order or amount of sequences delivered or both, or any combination thereof.

6. The method according to claim 5, wherein the tailoring is by the subject, an agent of the subject, or both.

7. The method according to claim 1, wherein the improving of the perception of wellness by the subject comprises reducing and/or alleviating a level of one or more negative feelings felt by a subject, invoking, increasing, and/or maintaining a level of one or more positive feelings felt by a subject; reducing and/or alleviating a negative mood felt by a subject, invoking, increasing, and/or maintaining a positive mood felt by a subject, inducing the prevalence or occurrence of one or more desirable physiological responses, reducing the prevalence or occurrence of one or more undesirable physiological responses, or any combination thereof.

8. The method according to claim 1 wherein:
(a) the at least one sensory stimulation comprises visual stimulation; or
(b) the at least one sensory stimulation comprises auditory stimulation; or
(c) the at least one sensory stimulation comprise somatic stimulation; or
(d) the at least one sensory stimulation comprises olfactory stimulation; or
(e) the at least one sensory stimulation comprises vestibular stimulation; or
(f) the at least one sensory stimulation comprises visual and auditory stimulation; or
(g) the at least one sensory stimulation comprises visual and somatic stimulation; or
(h) the at least one sensory stimulation comprises visual and olfactory stimulation; or
(i) the at least one sensory stimulation comprises visual and vestibular stimulation; or
(j) the at least one sensory stimulation comprises auditory and somatic stimulation; or
(k) the at least one sensory stimulation comprises auditory and olfactory stimulation; or
(l) the at least one sensory stimulation comprises auditory and vestibular stimulation; or
(m) the at least one sensory stimulation comprises somatic and olfactory stimulation; or
(n) the at least one sensory stimulation comprises somatic and vestibular stimulation; or
(o) the at least one sensory stimulation comprises olfactory and vestibular stimulation.

9. The method according to claim 8 wherein the mobile application is designed to deliver the at least one sensory stimulation via bilateral delivery.

10. The method according to claim 8 wherein the mobile application is designed to deliver the at least one sensory stimulation via intermittent bilateral delivery.

11. The method according to claim 8, wherein the mobile application is designed to deliver the sensory stimulation in an alternating pattern.
